(19) United States
(12) Patent Application Publication
(10) Pub. No.: US 2026/0347480 A1
(43) Pub. Date: Nov. 5, 2026

(54) ANTIBODIES BINDING TO CLEC12A

(71) Applicant: QLSF BIOTHERAPEUTICS, INC., South San Francisco, CA (US)

(72) Inventors: Mengyao JIN, South San Francisco, CA (US); Shihao CHEN, San Mateo, CA (US)

(21) Appl. No.: 19/099,676

(22) PCT Filed: Aug. 8, 2023
(86) PCT No.: PCT/US2023/071829

§ 371 Date: Jan. 29, 2025

Related U.S. Application Data
(60) Provisional application No. 63/396,536, filed on Aug. 9, 2022.

(51) Int. Cl. C07K 16/28; A61K 39/00; A61P 35/02

(52) U.S. Cl. CPC C07K 16/2851; A61P 35/02; C07K 16/2809; A61K 2039/505; C07K 2317/31

(57) ABSTRACT

Anti-CLEC12A antibodies are disclosed, along with methods of making such antibodies, compositions, including pharmaceutical compositions, comprising such antibodies, and their use to treat disorders that are characterized by the expression of CLEC12A.

### CROSS-REFERENCE TO RELATED APPLICATIONS

This application claims priority benefit to the filing date of U.S. Provisional Application No. 63/396,536, filed on Aug. 9, 2022, the disclosure of which is incorporated by reference herein in its entirety.

### FIELD OF THE INVENTION

The present invention concerns antibodies binding to CLEC12A. The invention further concerns methods of making such antibodies, compositions, including pharmaceutical compositions, comprising such antibodies, and their use to treat disorders that are characterized by the expression of CLEC12A.

### BACKGROUND OF THE INVENTION

### CLEC12A

CLEC12A, also known as C-type Lectin Domain Family 12 Member A, DCAL-2, CLL-1, MICL and CD371, is a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function.

RNA expression analysis from TCGA databases shows that CLEC12A is highly expressed and specific to acute myeloid leukemia. Therapeutic development of antibodies targeting CLEC12A may therefore be efficacious in treating AML patients, potentially including a significant number of AML patients who are refractory to current standard of care treatments.

### SUMMARY OF THE INVENTION

Aspects of the invention relate to CLEC12A-binding antibodies. Further aspects of the invention relate to methods of making such antibodies, compositions comprising such antibodies, and their use in the treatment of disorders that are characterized by the expression of CLEC12A.

In some embodiments, an antibody that binds to CLEC12A comprises a first binding unit comprising: a heavy chain variable region comprising: (a) a CDRH1 sequence of any one of SEQ ID NOs: 1-5; and/or (b) a CDRH2 sequence of any one of SEQ ID NOs: 6-14; and/or (c) a CDRH3 sequence of any one of SEQ ID NOs: 15-22; and a light chain variable region comprising: (d) a CDRL1 sequence of any one of SEQ ID NOs: 23-27; and/or (e) a CDRL2 sequence of any one of SEQ ID NOs: 28-33; and/or (f) a CDRL3 sequence of any one of SEQ ID NOs: 34-40. In some embodiments, the CDRH1, CDRH2 and CDRH3 sequences of the first binding unit are present in a human VH framework. In some embodiments, the CDRL1, CDRL2 and CDRL3 sequences of the first binding unit are present in a human VL framework.

In some embodiments, the first binding unit comprises: a heavy chain variable region comprising: (a) a CDRH1 sequence of any one of SEQ ID NOs: 1-5; and (b) a CDRH2 sequence of any one of SEQ ID NOs: 6-14; and (c) a CDRH3 sequence of any one of SEQ ID NOs: 15-22.

In some embodiments, the first binding unit comprises: a heavy chain variable region comprising: (a) a CDRH1 sequence of SEQ ID NO: 1, a CDRH2 sequence of SEQ ID NO: 6, and a CDRH3 sequence of SEQ ID NO: 15; or (b) a CDRH1 sequence of SEQ ID NO: 1, a CDRH2 sequence of SEQ ID NO: 7, and a CDRH3 sequence of SEQ ID NO: 16; or (c) a CDRH1 sequence of SEQ ID NO: 1, a CDRH2 sequence of SEQ ID NO: 8, and a CDRH3 sequence of SEQ ID NO: 17; or (d) a CDRH1 sequence of SEQ ID NO: 1, a CDRH2 sequence of SEQ ID NO: 9, and a CDRH3 sequence of SEQ ID NO: 18; or (e) a CDRH1 sequence of SEQ ID NO: 2, a CDRH2 sequence of SEQ ID NO: 10, and a CDRH3 sequence of SEQ ID NO: 19; or (f) a CDRH1 sequence of SEQ ID NO: 3, a CDRH2 sequence of SEQ ID NO: 11, and a CDRH3 sequence of SEQ ID NO: 20; or (g) a CDRH1 sequence of SEQ ID NO: 1, a CDRH2 sequence of SEQ ID NO: 12, and a CDRH3 sequence of SEQ ID NO: 21; or (h) a CDRH1 sequence of SEQ ID NO: 4, a CDRH2 sequence of SEQ ID NO: 13, and a CDRH3 sequence of SEQ ID NO: 22; or (i) a CDRH1 sequence of SEQ ID NO: 5, a CDRH2 sequence of SEQ ID NO: 14, and a CDRH3 sequence of SEQ ID NO: 22.

In some embodiments, the first binding unit comprises: a light chain variable region comprising: (a) a CDRL1 sequence of any one of SEQ ID NOs: 23-27; and (b) a CDRL2 sequence of any one of SEQ ID NOs: 28-33; and (c) a CDRL3 sequence of any one of SEQ ID NOs: 34-40. In some embodiments, the first binding unit comprises: a light chain variable region comprising: (a) a CDRL1 sequence of SEQ ID NO: 23, a CDRL2 sequence of SEQ ID NO: 28, and a CDRL3 sequence of SEQ ID NO: 34; or (b) a CDRL1 sequence of SEQ ID NO: 24, a CDRL2 sequence of SEQ ID NO: 29, and a CDRL3 sequence of SEQ ID NO: 35; or (c) a CDRL1 sequence of SEQ ID NO: 25, a CDRL2 sequence of SEQ ID NO: 30, and a CDRL3 sequence of SEQ ID NO: 36; or (d) a CDRL1 sequence of SEQ ID NO: 26, a CDRL2 sequence of SEQ ID NO: 31, and a CDRL3 sequence of SEQ ID NO: 37; or (e) a CDRL1 sequence of SEQ ID NO: 25, a CDRL2 sequence of SEQ ID NO: 30, and a CDRL3 sequence of SEQ ID NO: 38; or (f) a CDRL1 sequence of SEQ ID NO: 27, a CDRL2 sequence of SEQ ID NO: 32, and a CDRL3 sequence of SEQ ID NO: 39; or (g) a CDRL1 sequence of SEQ ID NO: 24, a CDRL2 sequence of SEQ ID NO: 33, and a CDRL3 sequence of SEQ ID NO: 40.

In some embodiments, the first binding unit comprises: (a) a heavy chain variable region comprising: a CDRH1 sequence of SEQ ID NO: 1, a CDRH2 sequence of SEQ ID NO: 6, and a CDRH3 sequence of SEQ ID NO: 15; and a light chain variable region comprising: a CDRL1 sequence of SEQ ID NO: 23, a CDRL2 sequence of SEQ ID NO: 28, and a CDRL3 sequence of SEQ ID NO: 34; or (b) a heavy chain variable region comprising: a CDRH1 sequence of SEQ ID NO: 1, a CDRH2 sequence of SEQ ID NO: 7, and a CDRH3 sequence of SEQ ID NO: 16; and a light chain variable region comprising: a CDRL1 sequence of SEQ ID NO: 23, a CDRL2 sequence of SEQ ID NO: 28, and a CDRL3 sequence of SEQ ID NO: 34; or (c) a heavy chain variable region comprising: a CDRH1 sequence of SEQ ID NO: 1, a CDRH2 sequence of SEQ ID NO: 8, and a CDRH3 sequence of SEQ ID NO: 17; and a light chain variable region comprising: a CDRL1 sequence of SEQ ID NO: 24, a CDRL2 sequence of SEQ ID NO: 29, and a CDRL3 sequence of SEQ ID NO: 35; or (d) a heavy chain variable region comprising: a CDRH1 sequence of SEQ ID NO: 1, a CDRH2 sequence of SEQ ID NO: 9, and a CDRH3 sequence of SEQ ID NO: 18; and a light chain variable region comprising: a CDRL1 sequence of SEQ ID NO: 25, a CDRL2 sequence of SEQ ID NO: 30, and a CDRL3 sequence of SEQ ID NO: 36; or (e) a heavy chain variable region comprising: a CDRH1 sequence of SEQ ID NO: 2, a CDRH2 sequence of SEQ ID NO: 10, and a CDRH3 sequence of SEQ ID NO: 19; and a light chain variable region comprising: a CDRL1 sequence of SEQ ID NO: 26, a CDRL2 sequence of SEQ ID NO: 31, and a CDRL3 sequence of SEQ ID NO: 37; or (f) a heavy chain variable region comprising: a CDRH1 sequence of SEQ ID NO: 1, a CDRH2 sequence of SEQ ID NO: 6, and a CDRH3 sequence of SEQ ID NO: 15; and a light chain variable region comprising: a CDRL1 sequence of SEQ ID NO: 23, a CDRL2 sequence of SEQ ID NO: 28, and a CDRL3 sequence of SEQ ID NO: 34; or (g) a heavy chain variable region comprising: a CDRH1 sequence of SEQ ID NO: 3, a CDRH2 sequence of SEQ ID NO: 11, and a CDRH3 sequence of SEQ ID NO: 20; and a light chain variable region comprising: a CDRL1 sequence of SEQ ID NO: 25, a CDRL2 sequence of SEQ ID NO: 30, and a CDRL3 sequence of SEQ ID NO: 38; or (h) a heavy chain variable region comprising: a CDRH1 sequence of SEQ ID NO: 1, a CDRH2 sequence of SEQ ID NO: 12, and a CDRH3 sequence of SEQ ID NO: 21; and a light chain variable region comprising: a CDRL1 sequence of SEQ ID NO: 27, a CDRL2 sequence of SEQ ID NO: 32, and a CDRL3 sequence of SEQ ID NO: 39; or (i) a heavy chain variable region comprising: a CDRH1 sequence of SEQ ID NO: 4, a CDRH2 sequence of SEQ ID NO: 13, and a CDRH3 sequence of SEQ ID NO: 22; and a light chain variable region comprising: a CDRL1 sequence of SEQ ID NO: 24, a CDRL2 sequence of SEQ ID NO: 33, and a CDRL3 sequence of SEQ ID NO: 40; or (j) a heavy chain variable region comprising: a CDRH1 sequence of SEQ ID NO: 5, a CDRH2 sequence of SEQ ID NO: 14, and a CDRH3 sequence of SEQ ID NO: 22; and a light chain variable region comprising: a CDRL1 sequence of SEQ ID NO: 24, a CDRL2 sequence of SEQ ID NO: 33, and a CDRL3 sequence of SEQ ID NO: 40.

In some embodiments, the first binding unit comprises a heavy chain variable region sequence having at least 95% identity to any one of SEQ ID NOs: 41-52. In some embodiments, the first binding unit comprises a heavy chain variable region sequence comprising any one of SEQ ID NOs: 41-52. In some embodiments, the first binding unit comprises a light chain variable region sequence having at least 95% identity to any one of SEQ ID NOs: 53-64. In some embodiments, the first binding unit comprises a light chain variable region sequence comprising any one of SEQ ID NOs: 53-64.

In some embodiments, the first binding unit comprises: (a) a heavy chain variable region sequence of SEQ ID NO: 41 and a light chain variable region sequence of SEQ ID NO: 53; or (b) a heavy chain variable region sequence of SEQ ID NO: 42 and a light chain variable region sequence of SEQ ID NO: 54; or (c) a heavy chain variable region sequence of SEQ ID NO: 43 and a light chain variable region sequence of SEQ ID NO: 55; or (d) a heavy chain variable region sequence of SEQ ID NO: 44 and a light chain variable region sequence of SEQ ID NO: 56; or (e) a heavy chain variable region sequence of SEQ ID NO: 45 and a light chain variable region sequence of SEQ ID NO: 57; or (f) a heavy chain variable region sequence of SEQ ID NO: 46 and a light chain variable region sequence of SEQ ID NO: 58; or (g) a heavy chain variable region sequence of SEQ ID NO: 47 and a light chain variable region sequence of SEQ ID NO: 59; or (h) a heavy chain variable region sequence of SEQ ID NO: 48 and a light chain variable region sequence of SEQ ID NO: 60; or (i) a heavy chain variable region sequence of SEQ ID NO: 49 and a light chain variable region sequence of SEQ ID NO: 61; or (j) a heavy chain variable region sequence of SEQ ID NO: 50 and a light chain variable region sequence of SEQ ID NO: 62; or (k) a heavy chain variable region sequence of SEQ ID NO: 51 and a light chain variable region sequence of SEQ ID NO: 63; or (1) a heavy chain variable region sequence of SEQ ID NO: 52 and a light chain variable region sequence of SEQ ID NO: 64.

In some embodiments, an antibody further comprises a heavy chain constant region. In some embodiments, the heavy chain constant region comprises a hinge region, a CH1 region, a CH2 region, and/or a CH3 region. In some embodiments, the heavy chain constant region comprises one or more knob-in-hole (KiH) mutations. In some embodiments, the heavy chain constant region comprises one or more silencing mutations. In some embodiments, the heavy chain constant region comprises one or more protein A binding mutations. In some embodiments, the one or more protein A binding mutations comprise an H435R mutation, a Y436F mutation, or both an H435R and a Y436F mutation.

In some embodiments, an antibody further comprises a light chain constant region. In some embodiments, the light chain constant region comprises a CL region. In some embodiments, an antibody is monospecific. In some embodiments, an antibody is multispecific. In some embodiments, an antibody is bispecific.

In some embodiments, an antibody further comprises a second binding unit that binds to CD38. In some embodiments, the second binding unit comprises: a heavy chain variable region comprising: (a) a CDRH1 sequence of SEQ ID NO: 65; and/or (b) a CDRH2 sequence of any one of SEQ ID NOs: 66-67; and/or (c) a CDRH3 sequence of SEQ ID NO: 68; and a light chain variable region comprising: (d) a CDRL1 sequence of SEQ ID NO: 69; and/or (e) a CDRL2 sequence of SEQ ID NO: 70; and/or (f) a CDRL3 sequence of SEQ ID NO: 71. In some embodiments, the CDRH1, CDRH2 and CDRH3 sequences of the second binding unit are present in a human VH framework. In some embodiments, the CDRL1, CDRL2 and CDRL3 sequences of the second binding unit are present in a human VL framework. In some embodiments, the second binding unit comprises: a heavy chain variable region comprising: (a) a CDRH1 sequence of SEQ ID NO: 65; and (b) a CDRH2 sequence of any one of SEQ ID NOs: 66-67; and (c) a CDRH3 sequence of SEQ ID NO: 68. In some embodiments, the second binding unit comprises: a heavy chain variable region comprising: (a) a CDRH1 sequence of SEQ ID NO: 65, a heavy chain CDRH2 sequence of SEQ ID NO: 66, and a heavy chain CDRH3 sequence of SEQ ID NO: 68; or (b) a CDRH1 sequence of SEQ ID NO: 65, a CDRH2 sequence of SEQ ID NO: 67, and a CDRH3 sequence of SEQ ID NO: 68.

In some embodiments, the second binding unit comprises: a light chain variable region comprising: a CDRL1 sequence of SEQ ID NO: 69; a CDRL2 sequence of SEQ ID NO: 70; and a CDRL3 sequence of SEQ ID NO: 71. In some embodiments, the second binding unit comprises: (a) a heavy chain variable region comprising: a CDRH1 sequence of SEQ ID NO: 65, a CDRH2 sequence of SEQ ID NO: 66, and a CDRH3 sequence of SEQ ID NO: 68; and a light chain variable region comprising: a CDRL1 sequence of SEQ ID NO: 69, a CDRL2 sequence of SEQ ID NO: 70, and a CDRL3 sequence of SEQ ID NO: 71; or (b) a heavy chain variable region comprising: a CDRH1 sequence of SEQ ID NO: 65, a CDRH2 sequence of SEQ ID NO: 67, and a CDRH3 sequence of SEQ ID NO: 68; and a light chain variable region comprising: a CDRL1 sequence of SEQ ID NO: 69, a CDRL2 sequence of SEQ ID NO: 70, and a CDRL3 sequence of SEQ ID NO: 71.

In some embodiments, the second binding unit comprises a heavy chain variable region sequence having at least 95% identity to any one of SEQ ID NOs: 72-73. In some embodiments, the second binding unit comprises a heavy chain variable region sequence comprising any one of SEQ ID NOs: 72-73. In some embodiments, the second binding unit comprises a light chain variable region sequence having at least 95% identity to any one of SEQ ID NOs: 74-75. In some embodiments, the second binding unit comprises a light chain variable region sequence comprising any one of SEQ ID NOs: 74-75.

In some embodiments, the second binding unit comprises: (a) a heavy chain variable region sequence of SEQ ID NO: 72 and a light chain variable region sequence of SEQ ID NO: 74; or (b) a heavy chain variable region sequence of SEQ ID NO: 73 and a light chain variable region sequence of SEQ ID NO: 75.

Aspects of the invention include an antibody that binds to CLEC12A and CD38, comprising: a first light chain subunit comprising SEQ ID NO: 79; a first heavy chain subunit comprising SEQ ID NO: 76; a second light chain subunit comprising SEQ ID NO: 79; and a second heavy chain subunit comprising SEQ ID NO: 78.

Aspects of the invention include an antibody that binds to CLEC12A and CD38, comprising: a first light chain subunit comprising SEQ ID NO: 79; a first heavy chain subunit comprising SEQ ID NO: 77; a second light chain subunit comprising SEQ ID NO: 79; and a second heavy chain subunit comprising SEQ ID NO: 78.

Aspects of the invention include an antibody that binds to CLEC12A and CD38, comprising: a first light chain subunit comprising SEQ ID NO: 83; a first heavy chain subunit comprising SEQ ID NO: 80; a second light chain subunit comprising SEQ ID NO: 83; and a second heavy chain subunit comprising SEQ ID NO: 82.

Aspects of the invention include an antibody that binds to CLEC12A and CD38, comprising: a first light chain subunit comprising SEQ ID NO: 83; a first heavy chain subunit comprising SEQ ID NO: 81; a second light chain subunit comprising SEQ ID NO: 83; and a second heavy chain subunit comprising SEQ ID NO: 82.

Aspects of the invention include pharmaceutical compositions comprising an antibody as described herein.

Aspects of the invention include methods of treatment, comprising administering to an individual in need an effective dose of an antibody or pharmaceutical composition as described herein.

Aspects of the invention include methods for the treatment of a disorder characterized by expression of CLEC12A, comprising administering to a subject with said disorder an antibody or a pharmaceutical composition as described herein.

Aspects of the invention include use of an antibody as described herein, in the preparation of a medicament for the treatment of a disorder characterized by expression of CLEC12A.

Aspects of the invention include an antibody as described herein, for use in the treatment of a disorder characterized by expression of CLEC12A.

In some embodiments, the disorder is a cancer. In some embodiments, the cancer is a blood cancer. In some embodiments, the blood cancer is acute myeloid leukemia (AML). In some embodiments, the AML is refractory AML.

Aspects of the invention include a polynucleotide encoding an antibody as described herein, a vector comprising such a polynucleotide, and a cell comprising such a vector.

Aspects of the invention include methods of producing an antibody as described herein, comprising growing a cell as described herein under conditions permissive for expression of the antibody, and isolating the antibody.

Aspects of the invention include kits comprising an antibody or a pharmaceutical composition as described herein, and instructions for use. In some embodiments, a kit further comprises an additional therapeutic agent.

These and further aspects will be further explained in the rest of the disclosure, including the Examples.

### BRIEF DESCRIPTION OF THE DRAWINGS

FIG. 1 is a table showing monovalent binding kinetics of humanized anti-CLEC12A antibodies in accordance with embodiments of the invention.

FIG. 2, Panels A-C, are a series of graphs showing binding data from humanized anti-CLEC12A antibodies binding to acute myeloid leukemia cells expressing CLEC12A.

FIG. 3, Panels A-D, are a series of graphs showing binding data from humanized anti-CLEC12A antibodies binding to human monocytes and neutrophils expressing CLEC12A.

FIG. 4 is a table showing EC50 values of humanized anti-CLEC12A antibodies binding to acute myeloid leukemia cell lines and primary human and cynomolgus monocytes and neutrophils expressing CLEC12A.

FIG. 5 is a schematic illustration of CLEC12A×CD3 multispecific antibody in accordance with embodiments of the invention.

FIG. 6, Panels A-C, are a series of graphs showing binding of bispecific CLEC12A×CD3 bispecific antibodies to acute myeloid leukemia cells expressing CLEC12A and to T-cells.

FIG. 7 is a graph showing binding of CLEC12A×CD3 bispecific antibodies to CLEC12A and CD3 on a sandwich ELISA.

FIG. 8, Panels A-B, are graphs showing proliferation of CD8+ T-cells mediated by CLEC12A x CD3 bispecific antibodies in accordance with embodiments of the invention, in the presence of autologous monocytes expressing CLEC12A.

FIG. 9, Panels A-D, are a series of graphs showing cytokine release from T-cells mediated by CLEC12A×CD3 bispecific antibodies in accordance with embodiments of the invention, in the presence of U937 cells expressing CLEC12A.

FIG. 10, Panels A-C, are a series of graphs showing CD8+ T-cell activation mediated by CLEC12A×CD3 bispecific antibodies in accordance with embodiments of the invention, in the presence of acute myeloid leukemia cells expressing CLEC12A. Panels D-F are a series of graphs showing corresponding EC50 values.

FIG. 11, Panels A-C, are a series of graphs showing cell killing of acute myeloid leukemia cells expressing CLEC12A mediated by CLEC12A×CD3 bispecific antibodies in accordance with embodiments of the invention, in the presence of CD8+ T-cells. Panels D-F are a series of graphs showing corresponding IC₅₀ values.

FIG. 12, Panels A-B, are graphs showing cell killing of CD14+ monocytes expressing CLEC12A mediated by CLEC12A×CD3 bispecific antibodies in accordance with embodiments of the invention, in the presence of autologous CD8+ T-cells.

FIG. 13 is a table showing a summary of IC₅₀ values for cell killing of CLEC12A positive cells.

FIG. 14 is a graph showing anti-tumor efficacy of CLEC12A×CD3 bispecific antibodies in accordance with embodiments of the invention, in a U937/human PBMC co-graft tumor model in NSG mice.

FIG. 15 is a graph showing anti-tumor efficacy of CLEC12A×CD3 bispecific antibodies in accordance with embodiments of the invention, in a U937/human PBMC reconstitution tumor model in NSG mice.

FIG. 16 is a table summarizing monovalent binding kinetics of CLEC12A×CD3 bispecific antibodies in accordance with embodiments of the invention to CLEC12A and CD3 epsilon.

FIG. 17 is a graph showing anti-tumor efficacy of CLEC12A×CD3 bispecific antibodies in accordance with embodiments of the invention in an orthotopic HL-60-Luc2 AML model.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VL) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or antigen-binding molecule (including multi-specific (e.g., bispecific) antibodies) is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signal transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain-only antibodies, three chain antibodies, TCAs, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule, or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). In some embodiments, “CDR” means a complementary determining region of an antibody as defined in Lefranc, M P et al., IMGT, the international ImMunoGene Tics database, Nucleic Acids Res., 27:209-212 (1999). “Framework Region” or “FR” residues are those variable domain residues other than the hypervariable region/CDR residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139., each of which is herein specifically incorporated by reference.

An “intact antibody chain” as used herein is one comprising a full-length variable region and a full-length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called K (kappa) and 2 (lambda), based on the amino acid sequences of their constant domains. Antibodies in accordance with embodiments of the invention can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563; Hezareh et al., (2001) J. Virology 75:12161; U.S. Pat. No. 5,624,821, the disclosures of which are incorporated herein by reference in their entireties). In some embodiments, a variant Fc sequence can include the following amino acid substitutions: L234A; L235A; and G237A. When these three amino acid substitutions are present in an IgG1 Fc sequence, they can be referred to as G1AAA.

Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour KL. et al., 1999 Eur J Immunol. 29 (8): 2613-24; and Shields R L. et al., 2001. J Biol Chem. 276 (9): 6591-604). The human IgG4 Fc amino acid sequence (UniProtKB No. P01861) is provided herein as SEQ ID NO: 76. Silenced IgG1 is described, for example, in Boesch, A. W., et al., “Highly parallel characterization of IgG Fc binding interactions.” MAbs, 2014. 6 (4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Further, an Fc variant can be constructed to facilitate heterodimerization of desired heavy chain polypeptide subunits, e.g., using knobs-into-holes, or KiH mutations. In some embodiments, a heavy chain polypeptide can include one or more “hole” mutations, such as, e.g., Y349C, T366S, L368A and/or Y407V, or any combination thereof. In some embodiments, a heavy chain polypeptide can include one or more “knob” mutations, such as, e.g., S354C and/or T366W, or any combination thereof. Antibodies in accordance with embodiments of the invention can utilize any suitable combination of knob and hole residues to facilitate desired heterodimer formation.

Fc variants can also be constructed to facilitate half-life extension, e.g., through enhanced FcRn binding. In some embodiments, a heavy chain polypeptide can include a T250Q mutation for this purpose. In some embodiments, a heavy chain polypeptide can include an M428L mutation for this purpose. In some embodiments, a heavy chain polypeptide can include a T250Q mutation and an M428L mutation for this purpose.

Fc variants can also be constructed to facilitate improved purification procedures, e.g., through enhanced protein A binding. In some embodiments, a heavy chain polypeptide can include an H435R mutation for this purpose. In some embodiments, a heavy chain polypeptide can include a Y436F mutation for this purpose. In some embodiments, a heavy chain polypeptide can include an H435R and a Y436F mutation for this purpose.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

Aspects of the invention include antibodies having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 84) (n=1) and GGGGSGGGGS (SEQ ID NO: 85) (n=2). Additional non-limiting examples of linkers include EPKSCDKTHT (SEQ ID NO: 86) and EPKSSDKTHT (SEQ ID NO: 87), which are derived from the natural hinge region of human IgG1. Other suitable linkers can also be used, and are described, for example, in Chen et al., Adv Drug Deliv Rev. 2013 Oct. 15; 65 (10): 1357-69, the disclosure of which is incorporated herein by reference in its entirety. Additional linker sequences may be described elsewhere herein, and can be incorporated into the subject antibodies in any suitable configuration.

Antibodies (e.g., multispecific antibodies) as described herein can be in the form of a dimer, in which two heavy chains are disulfide bonded or otherwise covalently or non-covalently attached to each other, and can optionally include an asymmetric interface between two or more of the CH domains to facilitate proper pairing between polypeptide chains (commonly referred to as a “knobs-into-holes” interface). Knobs into holes antibody engineering techniques for heavy chain heterodimerization are discussed, for example, in Ridgway et al., Protein Eng. 1996 July; 9(7):17-21, and U.S. Pat. No. 8,216,805, the disclosures of which are incorporated by reference herein in their entireties. An Fc region comprising an asymmetric interface can be referred to herein with the abbreviation “KiH”, meaning knobs-into-holes. For example, aspects of the invention include a variant Fc region sequence, such as a GIAAA sequence, that contains an asymmetric interface, and which is referred to herein as “GIAAA KiH”.

The term “CLEC12A” as used herein refers to C-type lectin domain family 12 member A, which is a cell surface receptor that modulates signaling cascades and mediates tyrosine phosphorylation of target MAP kinases. The term “CLEC12A” includes a CLEC12A protein of any human and non-human animal species, and specifically includes human CLEC12A as well as CLEC12A of non-human mammals.

The term “human CLEC12A” as used herein includes any variants, isoforms and species homologs of human CLEC12A (UniProt Q5QGZ9), regardless of its source or mode of preparation. Thus, “human CLEC12A” includes human CLEC12A naturally expressed by cells and CLEC12A expressed on cells transfected with the human CLEC12A gene.

The terms “anti-CLEC12A antibody,” “CLEC12A antibody,” “CLEC12A-binding antibody” are used herein interchangeably to refer to an antibody as hereinabove defined, immunospecifically binding to CLEC12A, including human CLEC12A, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The term “multi-specific antibody” is used herein in the broadest sense and cover all antibodies with more than one binding specificity. The multi-specific anti-CLEC12A antibodies of the present invention specifically include antibodies immunospecifically binding to two or more non-overlapping epitopes on a CLEC12A protein, such as a human CLEC12A (i.e., bivalent and biparatopic). The multi-specific anti-CLEC12A antibodies of the present invention also specifically include antibodies immunospecifically binding to an epitope on a CLEC12A protein, such as human CLEC12A and to an epitope on a different protein, such as, for example, a CD3 protein, such as human CD3 (i.e., bivalent and biparatopic). The multi-specific anti-CLEC12A antibodies of the present invention also specifically include antibodies immunospecifically binding to two or more non-overlapping or partially overlapping epitopes on a CLEC12A protein, such as a human CLEC12A protein, and to an epitope on a different protein, such as, for example, a CD3 protein, such as human CD3 protein (i.e., trivalent and biparatopic).

Antibodies of the invention include monospecific antibodies, having one binding specificity. Monospecific antibodies specifically include antibodies comprising a single binding specificity, as well as antibodies comprising more than one binding unit having the same binding specificity. The terms “monospecific antibody” is used herein in the broadest sense and covers all antibodies with one binding specificity. The monospecific anti-CLEC12A antibodies of the present invention specifically include antibodies immunospecifically binding to one epitope on a CLEC12A protein, such as a human CLEC12A (monovalent and monospecific). The monospecific anti-CLEC12A antibodies of the present invention also specifically include antibodies having more than one binding unit (e.g., multivalent antibodies) immunospecifically binding to an epitope on a CLEC12A protein, such as human CLEC12A. For example, a monospecific antibody in accordance with embodiments of the invention can include two variable regions, each comprising an antigen-binding domain, wherein each antigen-binding domain binds to the same epitope on a CLEC12A protein (i.e., bivalent and monospecific).

An “epitope” is the site on the surface of an antigen molecule to which a single antibody molecule binds. Generally, an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s). As noted above, the present invention specifically includes anti-CLEC12A antibodies with polyepitopic specificities, i.e., anti-CLEC12A antibodies binding to one or more non-overlapping epitopes on a CLEC12A protein, such as a human CLEC12A; and anti-CLEC12A antibodies binding to one or more epitopes on a CLEC12A protein and to an epitope on a different protein, such as, for example, a CD3 protein. The term “non-overlapping epitope(s)” or “non-competitive epitope(s)” of an antigen is defined herein to mean epitope(s) that are recognized by one member of a pair of antigen-specific antibodies but not the other member. Pairs of antibodies, or antigen-binding regions targeting the same antigen on a multi-specific antibody, recognizing non-overlapping epitopes, do not compete for binding to that antigen and are able to bind that antigen simultaneously.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “valent” as used herein refers to a specified number of binding sites in an antibody molecule.

A “monovalent” antibody has one binding site. Thus, a monovalent antibody is also monospecific.

A “multi-valent” antibody has two or more binding sites. Thus, the terms “bivalent”, “trivalent”, and “tetravalent” refer to the presence of two binding sites, three binding sites, and four binding sites, respectively. Thus, a bispecific antibody according to the invention is at least bivalent and may be trivalent, tetravalent, or otherwise multi-valent. A bivalent antibody in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

The term “chimeric antigen receptor” or “CAR” is used herein in the broadest sense to refer to an engineered receptor, which grafts a desired binding specificity (e.g., the antigen-binding region of a monoclonal antibody or other ligand) to membrane-spanning and intracellular-signaling domains. Typically, the receptor is used to graft the specificity of a monoclonal antibody onto a T-cell to create a chimeric antigen receptors (CAR). (J Natl Cancer Inst, 2015; 108(7):dvj439; and Jackson et al., Nature Reviews Clinical Oncology, 2016; 13:370-383). CAR-T cells are T-cells that have been genetically engineered to produce an artificial T-cell receptor for use in immunotherapy. In one embodiment, “CAR-T cell” means a therapeutic T-cell expressing a transgene encoding one or more chimeric antigen receptors comprised minimally of an extracellular domain, a transmembrane domain, and at least one cytosolic domain.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12 (8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “cancer”, “tumor”, “cancerous”, and “malignant” as used interchangeably herein refer to or describe the physiological condition in mammals that is typically characterized by unregulated cell growth.

The terms “hematologic cancer”, “hematologic malignancy” and “blood cancer” are used interchangeably herein to refer to cancers that begin in blood-forming tissues, including, but not limited to, bone marrow and/or cells of the immune system. In one embodiment, the leukemia is an acute myeloid leukemia (AML).

The term “characterized by expression of CLEC12A” broadly refers to any disease or disorder in which CLEC12A expression is associated with or involved with one or more pathological processes that are characteristic of the disease or disorder. Such disorders include, but are not limited to, acute myeloid leukemia (AML).

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10:29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

### II. Detailed Description

### Anti-CLEC12A Antibodies

The present invention provides a family of closely related antibodies that bind to human CLEC12A. The antibodies of this family comprise a set of CDRH sequences as defined herein and shown in Table 1 and a set of CDRL sequences as defined herein and showing in Table 2.

Anti-CLEC12A antibodies in accordance with embodiments of the invention are exemplified by the provided heavy chain variable region (VH) sequences set forth in Table 3, and the provided light chain variable region (VL) sequences set forth in Table 4. The family of antibodies provides a number of benefits that contribute to utility as clinically therapeutic agent(s). The antibodies include members with a range of binding affinities, allowing the selection of a specific sequence with a desired binding affinity.

TABLE 1
Anti-CLEC12A antibody CDRH amino acid sequences.

| Clone ID | CDRH1 | CDRH2 | CDRH3 |
|---|---|---|---|
| A14 | DYYMN (SEQ ID | VINPYNGVPNYSQKFKD | AREWFFYFDV (SEQ |
| | NO: 1) | (SEQ ID NO: 6) | ID NO: 15) |
| huA14 | DYYMN (SEQ ID | VINPYNGVPNYSQKFKD | AREWFFYFDV (SEQ |
| | NO: 1) | (SEQ ID NO: 6) | ID NO: 15) |
| huA14.QF | DYYMN (SEQ ID | VINPYQGVPNYSQKFKD | AREFFFYFDV (SEQ |
| | NO: 1) | (SEQ ID NO: 7) | ID NO: 16) |
| 2A5 | DYYMN (SEQ ID | IINPYNGASTYNQNFRG | PPYDGYFEGLEY |
| | NO: 1) | (SEQ ID NO: 8) | (SEQ ID NO: 17) |
| hu2A5 | DYYMN (SEQ ID | IINPYNGASTYNQNFRG | PPYDGYFEGLEY |
| | NO: 1) | (SEQ ID NO: 8) | (SEQ ID NO: 17) |
| 2F7 | DYYMN (SEQ ID | VVNPYNGGTSYNQKFKG | CREWFVAMDS |
| | NO: 1) | (SEQ ID NO: 9) | (SEQ ID NO: 18) |
| 5D8 | SYGVS (SEQ ID | VIWGDGNTNYHSALIS | SNWDLYYAMDY |
| | NO: 2) | (SEQ ID NO: 10) | (SEQ ID NO: 19) |
| 10C5 | DYYMN (SEQ ID | VINPYNGVPNYSQKFKD | AREWFFYFDV (SEQ |
| | NO: 1) | (SEQ ID NO: 6) | ID NO: 15) |
| 10D6 | DYYIN (SEQ ID | VVNPYNGVSSYNQKFKG | CREWFVALDN |
| | NO: 3) | (SEQ ID NO: 11) | (SEQ ID NO: 20) |
| 3G12 | DYYMN (SEQ ID | DIYPNNGDSSYNQNFKG | SDHYFDY (SEQ ID |
| | NO: 1) | (SEQ ID NO: 12) | NO: 21) |
| 1F12 | AHTMN (SEQ ID | VINPYNGGYWYNQKFKG | GGTTVVDWYFDV |
| | NO: 4) | (SEQ ID NO: 13) | (SEQ ID NO: 22) |
| 5E3 | GYTMN (SEQ ID | IINPYNGGYWYNQKFKG | GGTTVVDWYFDV |
| | NO: 5) | (SEQ ID NO: 14) | (SEQ ID NO: 22) |

TABLE 2
Anti-CLEC12A antibody CDRL amino acid sequences.

| Clone ID | CDRL1 | CDRL2 | CDRL3 |
|---|---|---|---|
| A14 | SASSSINYMH (SEQ | DTSKLPS (SEQ ID | QQWDSNPPT (SEQ |
| | ID NO: 23) | NO: 28) | ID NO: 34) |
| huA14 | SASSSINYMH (SEQ | DTSKLPS (SEQ ID | QQWDSNPPT (SEQ |
| | ID NO: 23) | NO: 28) | ID NO: 34) |
| huA14.QF | SASSSINYMH (SEQ | DTSKLPS (SEQ ID | QQWDSNPPT (SEQ |
| | ID NO: 23) | NO: 28) | ID NO: 34) |
| 2A5 | KASQDVSTAVA | SASYRYT (SEQ ID | QQHYFTPRT (SEQ |
| | (SEQ ID NO: 24) | NO: 29) | ID NO: 35) |
| hu2A5 | KASQDVSTAVA | SASYRYT (SEQ ID | QQHYFTPRT (SEQ |
| | (SEQ ID NO: 24) | NO: 29) | ID NO: 35) |
| 2F7 | SASSSVDYMH (SEQ | DTSKLAS (SEQ ID | QQWRSTPLT (SEQ |
| | ID NO: 25) | NO: 30) | ID NO: 36) |
| 5D8 | KASQDIHKYLS | RANRLAD (SEQ ID | LQYDEFPFT (SEQ |
| | (SEQ ID NO: 26) | NO: 31) | ID NO: 37) |
| 10C5 | SASSSINYMH (SEQ | DTSKLPS (SEQ ID | QQWDSNPPT (SEQ |
| | ID NO: 23) | NO: 28) | ID NO: 34) |
| 10D6 | SASSSVDYMH (SEQ | DTSKLAS (SEQ ID | QQWRSNPLT (SEQ |
| | ID NO: 25) | NO: 30) | ID NO: 38) |
| 3G12 | RTSGNIHYNLA | NAKTLAD (SEQ ID | QHFWSDPWT (SEQ |
| | (SEQ ID NO: 27) | NO: 32) | ID NO: 39) |
| 1F12 | KASQDVSTAVA | WASTRHT (SEQ ID | QQHYYAPLT (SEQ |
| | (SEQ ID NO: 24) | NO: 33) | ID NO: 40) |
| 5E3 | KASQDVSTAVA | WASTRHT (SEQ ID | QQHYYAPLT (SEQ |
| | (SEQ ID NO: 24) | NO: 33) | ID NO: 40) |

TABLE 3
Anti-CLEC12A heavy chain variable domain amino acid sequences.

| Clone ID | Heavy chain variable region sequence |
|---|---|
| A14 | EVQLQQSGPVLVKPGASVKMSCKASGYTFTDYYMNWMKQSPGKRPE |
| | WIGVINPYNGVPNYSQKFKDKARLTVDKSSSTAFMEFVSLTSDDSVVFY |
| | CVRAREWFFYFDVWGTGTTVTVSS (SEQ ID NO: 41) |
| huA14 | QVQLVQSGAEVKKPGASVKVSCKASGYTFTDYYMNWVRQAPGQGLE |
| | WMGVINPYNGVPNYSQKFKDRVTMTRDTSISTAYMELSRLRSDDTVV |
| | YYCVRAREWFFYFDVWGQGTLVTVSS (SEQ ID NO: 42) |
| huA14.QF | QVQLVQSGAEVKKPGASVKVSCKASGYTFTDYYMNWVRQAPGQGLE |
| | WMGVINPYNGVPQYSQKFKDRVTMTRDTSISTAYMELSRLRSDDTVV |
| | YYCVRAREFFFYFDVWGQGTLVTVSS (SEQ ID NO: 43) |
| 2A5 | EVQLQQSGPVLVKPGASVKMSCKASGYTLTDYYMNWVKQRHGKSLE |
| | WIGIINPYNGASTYNQNFRGKATLTVDRSSSTAYMELNSLTSEDSAVYY |
| | CARPPYDGYFEGLEYWGQGTSVTVSS (SEQ ID NO: 44) |
| hu2A5 | QVQLVQSGAEVKKPGASVKVSCKASGYTLTDYYMNWVRQAPGQGLE |
| | WMGIINPYNGASTYNQNFRGRVTMTRDTSTSTVYMELSSLRSEDTAVY |
| | YCARPPYDGYFEGLEYWGQGTLVTVSS (SEQ ID NO: 45) |
| 2F7 | EVQLQQSGPVLVKPGASVKMSCKSSGYTFTDYYMNWVKQSHGKSLE |
| | WIGVVNPYNGGTSYNQKFKGKATLTVDKSSSTAFMELNSLTSEDSAVY |
| | YCTRCREWFVAMDSWGQGTSVTVSA (SEQ ID NO: 46) |
| 5D8 | QVQLKESGPGLVAPSQSLSITCTVSGFSLNSYGVSWVRQPPGKGLEWLG |
| | VIWGDGNTNYHSALISRLSINKDNSKSQVFLKLNSLQTDDTATYYCAKS |
| | NWDLYYAMDYWGQGTSVTVSS (SEQ ID NO: 47) |
| 10C5 | EVQLQQSGPVLVKPGASVKMSCKASGYTFTDYYMNWMKQSPGKRPE |
| | WIGVINPYNGVPNYSQKFKDKARLTVDKSSSTAFMEFVSLTSDDSVVFY |
| | CVRAREWFFYFDVWGTGTTVTVSS (SEQ ID NO: 48) |
| 10D6 | EVQLQQSGPVLVKPGASVKMSCKSSGYTFTDYYINWVKQSHGESLEWI |
| | GVVNPYNGVSSYNQKFKGKATLTVDKSSSTAFMELSSLTSEDSAVYYC |
| | ARCREWFVALDNWGQGTSVTVSS (SEQ ID NO: 49) |
| 3G12 | EVQLQQSGPELVKPGASVNISCKASGYTFTDYYMNWVKQSHGKSLEWI |
| | GDIYPNNGDSSYNQNFKGKATLTVDRSSSTAYLDLRSLTSEDSAVYYC |
| | ARSDHYFDYWGQGTTLTVSS (SEQ ID NO: 50) |
| 1F12 | EVQLQQSGPELVKPGASMKISCRASGYSFTAHTMNWVKQSHGKNLEW |
| | IGVINPYNGGYWYNQKFKGKATLTVDKSSNTAYMELLSLTSEDSGVYY |
| | CARGGTTVVDWYFDVWGAGTTVTVSS (SEQ ID NO: 51) |
| 5E3 | EVQLQQSGPELVKPGASMKISCKASGYSFTGYTMNWVKQSHGKNLEW |
| | IGIINPYNGGYWYNQKFKGKATLTVDKSSNTAYMELLSLTSEDSGVYY |
| | CARGGTTVVDWYFDVWGAGTTVTVSS (SEQ ID NO: 52) |

TABLE 4
Anti-CLEC12A light chain variable domain amino acid sequences.

| Clone ID | Light chain variable region sequence |
|---|---|
| A14 | QIVLTQSPAIMSASPGEKVTMTCSASSSINYMHWYQQKSGTSPKRWIFDTSKLP |
| | SGVPSRFGGSGSGTSYSLTISSMEAEDAATYYCQQWDSNPPTFGGGTKLEIK |
| | (SEQ ID NO: 53) |
| huA14 | DIQMTQSPSSLSASVGDRVTITCSASSSINYMHWYQQKPGKAPKLLIYDTSKLPS |
| | GVPSRFSGSGSGTDFTLTISSLQPEDFATYYCQQWDSNPPTFGGGTKVEIK (SEQ |
| | ID NO: 54) |
| huA14.QF | DIQMTQSPSSLSASVGDRVTITCSASSSINYMHWYQQKPGKAPKLLIYDTSKLPS |
| | GVPSRFSGSGSGTDFTLTISSLQPEDFATYYCQQWDSNPPTFGGGTKVEIK (SEQ |
| | ID NO: 55) |
| 2A5 | DIVMTQSHKFLSSSVGDRVSLTCKASQDVSTAVAWYQQKPGQSPKLLIYSASY |
| | RYTGVPDRFTGSGSGTDFTFTISSVQAEDLAVYYCQQHYFTPRTFGGGTKLEIK |
| | (SEQ ID NO: 56) |
| hu2A5 | DIQMTQSPSSLSASVGDRVTITCKASQDVSTAVAWYQQKPGKAPKLLIYSASYR |
| | YTGVPSRFSGSGSGTDFTLTISSLQPEDFATYYCQQHYFTPRTFGGGTKVEIK |
| | (SEQ ID NO: 57) |
| 2F7 | QIVLTQSPAIMSASPGEKVTMTCSASSSVDYMHWYQQKSGTSPKRWIYDTSKL |
| | ASGVPARFSGSGSGTSYSLTISSMEAEDAATYYCQQWRSTPLTFGGGTKLELK |
| | (SEQ ID NO: 58) |
| 5D8 | DIKMTQSPSSMYASLGERVTITCKASQDIHKYLSWFQQKSGTSPKTLIYRANRL |
| | ADGVPSRFSGSGSGQDWSLTISSLEYEDLGIYYCLQYDEFPFTFGAGTKLEIK |
| | (SEQ ID NO: 59) |
| 10C5 | QIVLTQSPAIMSASPGEKVTMTCSASSSINYMHWYQQKSGTSPKRWIFDTSKLP |
| | SGVPSRFGGSGSGTSYSLTISSMEAEDAATYYCQQWDSNPPTFGGGTKLEIK |
| | (SEQ ID NO: 60) |
| 10D6 | QIVLTQSPAIMSASPGEKVTMTCSASSSVDYMHWYQQKSGTSPKRWIYDTSKL |
| | ASGVPARFSGSGSGTSYSLTISSMEAEDAATYYCQQWRSNPLTFGAGTKLELK |
| | (SEQ ID NO: 61) |
| 3G12 | DIQMTQSPASLSASVGETVTITCRTSGNIHYNLAWYQQKQGKSPQLLVYNAKT |
| | LADGVPSRFSGSGSGTLYSLNINSLQPEDFGSYYCQHFWSDPWTFGGGTKLEIT |
| | (SEQ ID NO: 62) |
| 1F12 | DIVMTQSHKFMSTSVGDRVSITCKASQDVSTAVAWYQQKTGQSPKLLIYWAST |
| | RHTGVPDRFTGSGSGTDYSFTISSVQAEDLALYYCQQHYYAPLTFGAGTKLELK |
| | (SEQ ID NO: 63) |
| 5E3 | DIVMTQSHKFMSTSVGDRVSITCKASQDVSTAVAWYQQKPGQSPKLLIYWAST |
| | RHTGVPDRFTGSGSGTDYSFTISSVQAEDLALYYCQQHYYAPLTFGAGTKLELK |
| | (SEQ ID NO: 64) |

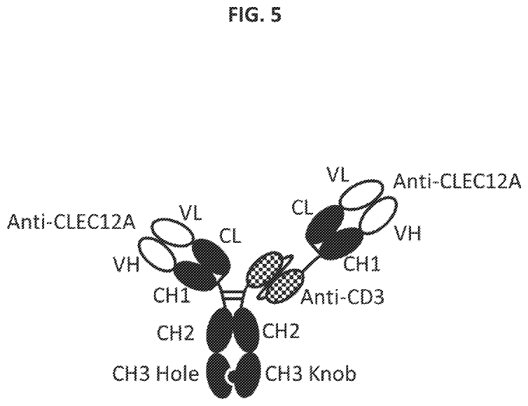

A suitable antibody may be selected from those provided herein for development and therapeutic or other use, including, without limitation, use as a bispecific antibody, e.g., as shown in FIG. 5, or part of a CAR-T structure. FIG. 5 is a schematic illustration of anti-CLEC12A x anti-CD38 multi-specific antibody, where the anti-CLEC12A binding regions are located at the N-termini of the polypeptide subunits, and the CD3& binding unit is in an scFv format, and is located on one of the heavy chain polypeptide subunits between the variable region and the hinge region. In some embodiments, the two heavy chain polypeptide subunits of a heterodimeric mutltispecific antibody are pared using, e.g., knobs-into-holes (KiH) technology.

Determination of affinity for a candidate protein can be performed using methods known in the art, such as Biacore measurements. Members of the antibody family may have an affinity for CLEC12A with a Kd of from about 10-6 to around about 10-11, including without limitation: from about 10-6 to around about 10-10; from about 10-6 to around about 10-9; from about 10-6 to around about 10-8; from about 10-8 to around about 10-11; from about 10-8 to around about 10-10; from about 10-8 to around about 10-9; from about 10-9 to around about 10-11; from about 10-9 to around about 10-10; or any value within these ranges. The affinity selection may be confirmed with a biological assessment for modulating, e.g., blocking, a CLEC12A biological activity, including in vitro assays, pre-clinical models, and clinical trials, as well as assessment of potential toxicity.

Members of the antibody family herein are cross-reactive with the CLEC12A protein of Cynomolgus macaque, and can be engineered to provide cross-reactivity with the CLEC12A protein of any other animal species, if desired.

The family of anti-CLEC12A antibodies herein comprises one or more binding units comprising a VH domain, comprising CDRH1, CDRH2 and CDRH3 sequences in a human VH framework, and CDRL1, CDRL2 and CDRL3 sequences in a human Vkappa or Vlambda framework. The CDR sequences may be situated, as an example, in the region of around amino acid residues 26-33; 51-58; and 97-116 for CDR1, CDR2 and CDR3, respectively, of the provided exemplary variable region sequences set forth in Tables 3 and 4. It will be understood by one of ordinary skill in the art that the CDR sequences may be in different positions if a different framework sequence is selected, although generally the order of the sequences will remain the same.

In some embodiments, an anti-CLEC12A antibody comprises an CDRH1 sequence of any one of SEQ ID NOs: 1-5. In some embodiments, an anti-CLEC12A antibody comprises an CDRH2 sequence of any one of SEQ ID NOs: 6-14. In some embodiments, an anti-CLEC12A antibody comprises an CDRH3 sequence of any one of SEQ ID NOs: 15-22.

In one preferred embodiment, an anti-CLEC12A antibody comprises an CDRH1 sequence of SEQ ID NO: 1, an CDRH2 sequence of SEQ ID NO: 6, and an CDRH3 sequence of SEQ ID NO: 15. In one preferred embodiment, an anti-CLEC12A antibody comprises an CDRH1 sequence of SEQ ID NO: 1, an CDRH2 sequence of SEQ ID NO: 7, and an CDRH3 sequence of SEQ ID NO: 16. In one preferred embodiment, an anti-CLEC12A antibody comprises an CDRH1 sequence of SEQ ID NO: 1, an CDRH2 sequence of SEQ ID NO: 8, and an CDRH3 sequence of SEQ ID NO: 17. In one preferred embodiment, an anti-CLEC12A antibody comprises an CDRH1 sequence of SEQ ID NO: 1, an CDRH2 sequence of SEQ ID NO: 9, and an CDRH3 sequence of SEQ ID NO: 18. In one preferred embodiment, an anti-CLEC12A antibody comprises an CDRH1 sequence of SEQ ID NO: 2, an CDRH2 sequence of SEQ ID NO: 10, and an CDRH3 sequence of SEQ ID NO: 19. In one preferred embodiment, an anti-CLEC12A antibody comprises an CDRH1 sequence of SEQ ID NO: 3, an CDRH2 sequence of SEQ ID NO: 11, and an CDRH3 sequence of SEQ ID NO: 20. In one preferred embodiment, an anti-CLEC12A antibody comprises an CDRH1 sequence of SEQ ID NO: 1, an CDRH2 sequence of SEQ ID NO: 12, and an CDRH3 sequence of SEQ ID NO: 21. In one preferred embodiment, an anti-CLEC12A antibody comprises an CDRH1 sequence of SEQ ID NO: 4, an CDRH2 sequence of SEQ ID NO: 13, and an CDRH3 sequence of SEQ ID NO: 22. In one preferred embodiment, an anti-CLEC12A antibody comprises an CDRH1 sequence of SEQ ID NO: 5, an CDRH2 sequence of SEQ ID NO: 14, and an CDRH3 sequence of SEQ ID NO: 22.

In some embodiments, an anti-CLEC12A antibody comprises an CDRL1 sequence of any one of SEQ ID NOs: 23-27. In some embodiments, an anti-CLEC12A antibody comprises an CDRL2 sequence of any one of SEQ ID NOs: 28-33. In some embodiments, an anti-CLEC12A antibody comprises an CDRL3 sequence of any one of SEQ ID NOs: 34-40.

In one preferred embodiment, an anti-CLEC12A antibody comprises an CDRL1 sequence of SEQ ID NO: 23, an CDRL2 sequence of SEQ ID NO: 28, and an CDRL3 sequence of SEQ ID NO: 34. In one preferred embodiment, an anti-CLEC12A antibody comprises an CDRL1 sequence of SEQ ID NO: 24, an CDRL2 sequence of SEQ ID NO: 29, and an CDRL3 sequence of SEQ ID NO: 35. In one preferred embodiment, an anti-CLEC12A antibody comprises an CDRL1 sequence of SEQ ID NO: 25, an CDRL2 sequence of SEQ ID NO: 30, and an CDRL3 sequence of SEQ ID NO: 36. In one preferred embodiment, an anti-CLEC12A antibody comprises an CDRL1 sequence of SEQ ID NO: 26, an CDRL2 sequence of SEQ ID NO: 31, and an CDRL3 sequence of SEQ ID NO: 37. In one preferred embodiment, an anti-CLEC12A antibody comprises an CDRL1 sequence of SEQ ID NO: 25, an CDRL2 sequence of SEQ ID NO: 30, and an CDRL3 sequence of SEQ ID NO: 38. In one preferred embodiment, an anti-CLEC12A antibody comprises an CDRL1 sequence of SEQ ID NO: 27, an CDRL2 sequence of SEQ ID NO: 32, and an CDRL3 sequence of SEQ ID NO: 39. In one preferred embodiment, an anti-CLEC12A antibody comprises an CDRL1 sequence of SEQ ID NO: 24, an CDRL2 sequence of SEQ ID NO: 33, and an CDRL3 sequence of SEQ ID NO: 40.

In one preferred embodiment, an anti-CLEC12A antibody comprises a heavy chain variable region comprising: a CDRH1 sequence of SEQ ID NO: 1, a CDRH2 sequence of SEQ ID NO: 6, and a CDRH3 sequence of SEQ ID NO: 15; and a light chain variable region comprising: a CDRL1 sequence of SEQ ID NO: 23, a CDRL2 sequence of SEQ ID NO: 28, and a CDRL3 sequence of SEQ ID NO: 34.

In one preferred embodiment, an anti-CLEC12A antibody comprises a heavy chain variable region comprising: a CDRH1 sequence of SEQ ID NO: 1, a CDRH2 sequence of SEQ ID NO: 7, and a CDRH3 sequence of SEQ ID NO: 16; and a light chain variable region comprising: a CDRL1 sequence of SEQ ID NO: 23, a CDRL2 sequence of SEQ ID NO: 28, and a CDRL3 sequence of SEQ ID NO: 34.

In one preferred embodiment, an anti-CLEC12A antibody comprises a heavy chain variable region comprising: a CDRH1 sequence of SEQ ID NO: 1, a CDRH2 sequence of SEQ ID NO: 8, and a CDRH3 sequence of SEQ ID NO: 17; and a light chain variable region comprising: a CDRL1 sequence of SEQ ID NO: 24, a CDRL2 sequence of SEQ ID NO: 29, and a CDRL3 sequence of SEQ ID NO: 35.

In one preferred embodiment, an anti-CLEC12A antibody comprises a heavy chain variable region comprising: a CDRH1 sequence of SEQ ID NO: 1, a CDRH2 sequence of SEQ ID NO: 9, and a CDRH3 sequence of SEQ ID NO: 18; and a light chain variable region comprising: a CDRL1 sequence of SEQ ID NO: 25, a CDRL2 sequence of SEQ ID NO: 30, and a CDRL3 sequence of SEQ ID NO: 36.

In one preferred embodiment, an anti-CLEC12A antibody comprises a heavy chain variable region comprising: a CDRH1 sequence of SEQ ID NO: 2, a CDRH2 sequence of SEQ ID NO: 10, and a CDRH3 sequence of SEQ ID NO: 19; and a light chain variable region comprising: a CDRL1 sequence of SEQ ID NO: 26, a CDRL2 sequence of SEQ ID NO: 31, and a CDRL3 sequence of SEQ ID NO: 37.

In one preferred embodiment, an anti-CLEC12A antibody comprises a heavy chain variable region comprising: a CDRH1 sequence of SEQ ID NO: 1, a CDRH2 sequence of SEQ ID NO: 6, and a CDRH3 sequence of SEQ ID NO: 15; and a light chain variable region comprising: a CDRL1 sequence of SEQ ID NO: 23, a CDRL2 sequence of SEQ ID NO: 28, and a CDRL3 sequence of SEQ ID NO: 34.

In one preferred embodiment, an anti-CLEC12A antibody comprises a heavy chain variable region comprising: a CDRH1 sequence of SEQ ID NO: 3, a CDRH2 sequence of SEQ ID NO: 11, and a CDRH3 sequence of SEQ ID NO: 20; and a light chain variable region comprising: a CDRL1 sequence of SEQ ID NO: 25, a CDRL2 sequence of SEQ ID NO: 30, and a CDRL3 sequence of SEQ ID NO: 38.

In one preferred embodiment, an anti-CLEC12A antibody comprises a heavy chain variable region comprising: a CDRH1 sequence of SEQ ID NO: 1, a CDRH2 sequence of SEQ ID NO: 12, and a CDRH3 sequence of SEQ ID NO: 21; and a light chain variable region comprising: a CDRL1 sequence of SEQ ID NO: 27, a CDRL2 sequence of SEQ ID NO: 32, and a CDRL3 sequence of SEQ ID NO: 39.

In one preferred embodiment, an anti-CLEC12A antibody comprises a heavy chain variable region comprising: a CDRH1 sequence of SEQ ID NO: 4, a CDRH2 sequence of SEQ ID NO: 13, and a CDRH3 sequence of SEQ ID NO: 22; and a light chain variable region comprising: a CDRL1 sequence of SEQ ID NO: 24, a CDRL2 sequence of SEQ ID NO: 33, and a CDRL3 sequence of SEQ ID NO: 40.

In one preferred embodiment, an anti-CLEC12A antibody comprises a heavy chain variable region comprising: a CDRH1 sequence of SEQ ID NO: 5, a CDRH2 sequence of SEQ ID NO: 14, and a CDRH3 sequence of SEQ ID NO: 22; and a light chain variable region comprising: a CDRL1 sequence of SEQ ID NO: 24, a CDRL2 sequence of SEQ ID NO: 33, and a CDRL3 sequence of SEQ ID NO: 40.

In some embodiments, an anti-CLEC12A antibody comprises any of the heavy chain variable region amino acid sequences of SEQ ID NOs: 41-52 (Table 3). In some embodiments, an anti-CLEC12A antibody comprises a heavy chain variable region amino acid sequence that has at least about 80% identity, such as about 85%, 90%, 95%, 99% or 100% identity to the sequence of any one of SEQ ID NOs: 41-52 (Table 3).

In some embodiments, an anti-CLEC12A antibody comprises any of the light chain variable region amino acid sequences of SEQ ID NOs: 53-64 (Table 4). In some embodiments, an anti-CLEC12A antibody comprises a light chain variable region amino acid sequence that has at least about 80% identity, such as about 85%, 90%, 95%, 99% or 100% identity to the sequence of any one of SEQ ID NOs: 53-64 (Table 4).

In some embodiments, an anti-CLEC12A antibody comprises a heavy chain variable region sequence of SEQ ID NO: 41 and a light chain variable region sequence of SEQ ID NO: 53. In some embodiments, an anti-CLEC12A antibody comprises a heavy chain variable region sequence of SEQ ID NO: 42 and a light chain variable region sequence of SEQ ID NO: 54. In some embodiments, an anti-CLEC12A antibody comprises a heavy chain variable region sequence of SEQ ID NO: 43 and a light chain variable region sequence of SEQ ID NO: 55. In some embodiments, an anti-CLEC12A antibody comprises a heavy chain variable region sequence of SEQ ID NO: 44 and a light chain variable region sequence of SEQ ID NO: 56. In some embodiments, an anti-CLEC12A antibody comprises a heavy chain variable region sequence of SEQ ID NO: 45 and a light chain variable region sequence of SEQ ID NO: 57. In some embodiments, an anti-CLEC12A antibody comprises a heavy chain variable region sequence of SEQ ID NO: 46 and a light chain variable region sequence of SEQ ID NO: 58. In some embodiments, an anti-CLEC12A antibody comprises a heavy chain variable region sequence of SEQ ID NO: 47 and a light chain variable region sequence of SEQ ID NO: 59. In some embodiments, an anti-CLEC12A antibody comprises a heavy chain variable region sequence of SEQ ID NO: 48 and a light chain variable region sequence of SEQ ID NO: 60. In some embodiments, an anti-CLEC12A antibody comprises a heavy chain variable region sequence of SEQ ID NO: 49 and a light chain variable region sequence of SEQ ID NO: 61. In some embodiments, an anti-CLEC12A antibody comprises a heavy chain variable region sequence of SEQ ID NO: 50 and a light chain variable region sequence of SEQ ID NO: 62. In some embodiments, an anti-CLEC12A antibody comprises a heavy chain variable region sequence of SEQ ID NO: 51 and a light chain variable region sequence of SEQ ID NO: 63. In some embodiments, an anti-CLEC12A antibody comprises a heavy chain variable region sequence of SEQ ID NO: 52 and a light chain variable region sequence of SEQ ID NO: 64.

In some embodiments, a CDR sequence in an anti-CLEC12A antibody of the invention comprises one or two amino acid substitutions relative to a CDR1, CDR2 and/or CDR3 sequence or set of CDR1, CDR2 and CDR3 sequences in Table 1 or 2.

In some embodiments, an anti-CLEC12A antibody preferably comprises a heavy chain variable domain (VH) in which the CDRH3 sequence has greater than or equal to 80%, such as at least 85%, at least 90%, at least 95%, or at least 99% sequence identity at the amino acid level to a CDRH3 sequence of any one of the antibodies whose CDRH3 sequences are provided in Table 1, and binds to CLEC12A.

In some embodiments, an anti-CLEC12A antibody preferably comprises a heavy chain variable domain (VH) in which the full set of CDRHs 1, 2, and 3 (combined) has greater than or equal to eighty-five percent (85%) sequence identity at the amino acid level to the CDRHs 1, 2, and 3 (combined) of the antibodies whose CDRH sequences are provided in Table 1, and binds to CLEC12A.

In some embodiments, bispecific or multi-specific antibodies are provided, which may have any of the configurations discussed herein, including, without limitation, multispecific antibodies comprising one or more binding units located on a heavy chain polypeptide subunit, and placed in between an N-terminal binding unit and the hinge region, as shown, for example, in FIG. 5. In some embodiments, such a binding unit can comprise an scFv configuration, wherein a VH and VL region are connected by a linker sequence, and together form a binding unit that binds to a target protein, such, for example, CD3.

In some embodiments, a multi-specific antibody can comprise a first binding unit that binds to CLEC12A, comprising a heavy chain variable region paired with a light chain variable region, and a second binding unit that binds to a protein other than CLEC12A, comprising a heavy chain variable region paired with a light chain variable region. In some embodiments, a multispecific antibody can comprise first and second binding unit, each of which comprise a heavy chain variable region paired with a light chain variable region, and both of which bind to CLEC12A, and at least a third binding unit that binds to a protein other than CLEC12A, such as, for example, CD3. In some embodiments, the third binding unit is positioned at a C-terminus of one of the light chain polypeptide subunits that make up the antibody. In some embodiments, the third binding unit is positioned within one of the heavy chain polypeptide subunits, e.g., between the variable region and the hinge region of the polypeptide subunit, as depicted, for example, in FIG. 5. In some embodiments, the third binding unit comprises an scFv, wherein a heavy chain variable region is connected to a light chain variable region with a linker sequence.

In some embodiments, the anti-CLEC12A antibodies described herein comprise heavy chain constant region sequences, such as CH1, hinge, CH2, CH3 and/or CH4 domains. In some embodiments, the anti-CLEC12A antibodies described herein comprise light chain constant region sequences, such as CL domains. In some embodiments, the anti-CLEC12A antibodies described herein comprise heavy chain constant region sequences that form an Fc region.

As noted above, in some embodiments, a multi-specific anti-CLEC12A antibody comprises a binding unit that binds to a protein other than CLEC12A, such as, for example, CD3 (e.g., CD38). In some embodiments, a CD3 binding unit comprises a set of CDRH sequences as defined herein and shown in Table 5, and a set of CDRL sequences as defined herein and showing in Table 6.

TABLE 5
CD3 heavy chain CDR sequences:

| Clone | | | |
|---|---|---|---|
| ID | CDRH1 | CDRH2 | CDRH3 |
| 1B4 | TYAMN | RIRSKYNNYATYYADSVKD | HGNFGNSYVSWFAY |
| | (SEQ ID | (SEQ ID NO: 66) | (SEQ ID NO: 68) |
| | NO: 65) | | |
| 1B10 | TYAMN | RIRSKANNYATYYADSVKG | HGNFGNSYVSWFAY |
| | (SEQ ID | (SEQ ID NO: 67) | (SEQ ID NO: 68) |
| | NO: 65) | | |

TABLE 6
CD3 light chain CDR sequences:

| Clone | | | |
|---|---|---|---|
| ID | CDRL1 | CDRL2 | CDRL3 |
| 1B4 | GSSTGAVTTSNYAN | GTNKRAP | ALWYSNLWV |
| | (SEQ ID NO: 69) | (SEQ ID | (SEQ ID NO: 71) |
| | | NO: 70) | |
| 1B10 | GSSTGAVTTSNYAN | GTNKRAP | ALWYSNLWV |
| | (SEQ ID NO: 69) | (SEQ ID | (SEQ ID NO: 71) |
| | | NO: 70) | |

In some embodiments, a CD3 binding unit comprises a heavy chain variable region (VH) sequence set forth in Table 7, and a light chain variable region (VL) sequence set forth in Table 8. These CD3 binding units provide a number of benefits that contribute to utility as clinically therapeutic agent(s).

TABLE 7
CD3 heavy chain variable region sequences

| Clone | |
|---|---|
| ID | Heavy chain variable region sequence |
| 1B4 | EVKLVESGGGLVQPGGSLKLSCAASGFTFNTYAMNWVRQAP |
| | GKGLEWVGRIRSKYNNYATYYADSVKDRFTISRDDSQNILY |
| | LQMNNLRTEDTAVYYCVRHGNFGNSYVSWFAYWGQGTLVTV |
| | SS (SEQ ID NO: 72) |
| 1B10 | EVKLLESGGGLVQPGGSLKLSCAASGFTFNTYAMNWVRQAP |
| | GKGLEWVGRIRSKANNYATYYADSVKGRFTISRDDSKNTLY |
| | LQMNNLKAEDTAVYYCVRHGNFGNSYVSWFAYWGQGTLVTV |
| | SS (SEQ ID NO: 73) |

TABLE 8
CD3 light chain variable region sequences

| Clone | |
|---|---|
| ID | Light chain variable region sequence |
| 1B4 | QAFVTQEPSLTVSPGGTVTLTCGSSTGAVTTSNYANWVQQK |
| | PGQAPRGLIGGTNKRAPGVPARFSGSLIGDKAALTITGAQP |
| | EDEAEYYCALWYSNLWVFGGGTKLTVL (SEQ ID NO: |
| | 74) |
| 1B10 | EAVVTQEPSLTVSPGGTVTLTCGSSTGAVTTSNYANWVQQK |
| | PGQAPRGLIGGTNKRAPGTPARFSGSLLGDKAALTITGAQP |
| | EDEAEYYCALWYSNLWVFGGGTKLTVL (SEQ ID NO: |
| | 75) |

Multi-specific anti-CLEC12A antibodies in accordance with embodiments of the invention can include one or more CD3 binding unit, as described herein. Any of the binding domains of the anti-CLEC12A antibodies described herein can be combined with the CD3-binding domains described herein to generate multi-specific antibodies that bind to CLEC12A and CD3. Full length sequences that can be used to compose an anti-CLEC12A×CD3 antibody are provided in Table 9.

TABLE 9
CLEC12A × CD3 antibody full length polypeptide subunit sequences

| Name | Sequence |
|---|---|
| huA14.QF_1B10_G1AAA_knob_RF | QVQLVQSGAEVKKPGASVKVSCKASGYTFTDYYMN |
| | WVRQAPGQGLEWMGVINPYNGVPQYSQKFKDRVTM |
| | TRDTSISTAYMELSRLRSDDTVVYYCVRAREFFFYFDV |
| | WGQGTLVTVSSASTKGPSVFPLAPSSKSTSGGTAALGC |
| | LVKDYFPEPVTVSWNSGALTSGVHTFPAVLQSSGLYS |
| | LSSVVTVPSSSLGTQTYICNVNHKPSNTKVDKKVEPKS |
| | CDKTHTEVKLLESGGGLVQPGGSLKLSCAASGFTFNT |
| | YAMNWVRQAPGKGLEWVGRIRSKANNYATYYADSV |
| | KGRFTISRDDSKNTLYLQMNNLKAEDTAVYYCVRHG |
| | NFGNSYVSWFAYWGQGTLVTVSSGGGGSGGGGSGG |
| | GGSAEAVVTQEPSLTVSPGGTVTLTCGSSTGAVTTSNY |
| | ANWVQQKPGQAPRGLIGGTNKRAPGTPARFSGSLLGD |
| | KAALTITGAQPEDEAEYYCALWYSNLWVFGGGTKLT |
| | VLEPKSSDKTHTCPPCPAPEAAGAPSVFLFPPKPKDTL |
| | MISRTPEVTCVVVDVSHEDPEVKFNWYVDGVEVHNA |
| | KTKPREEQYNSTYRVVSVLTVLHQDWLNGKEYKCKV |
| | SNKALPAPIEKTISKAKGQPREPQVYTLPPCRDELTKN |
| | QVSLWCLVKGFYPSDIAVEWESNGQPENNYKTTPPVL |
| | DSDGSFFLYSKLTVDKSRWQQGNVFSCSVMHEALHN |
| | RFTQKSLSLSPG (SEQ ID NO: 76) |
| huA14.QF_1B4_G1AAA_knob_RF | QVQLVQSGAEVKKPGASVKVSCKASGYTFTDYYMN |
| | WVRQAPGQGLEWMGVINPYNGVPQYSQKFKDRVTM |
| | TRDTSISTAYMELSRLRSDDTVVYYCVRAREFFFYFDV |
| | WGQGTLVTVSSASTKGPSVFPLAPSSKSTSGGTAALGC |
| | LVKDYFPEPVTVSWNSGALTSGVHTFPAVLQSSGLYS |
| | LSSVVTVPSSSLGTQTYICNVNHKPSNTKVDKKVEPKS |
| | CDKTHTEVKLVESGGGLVQPGGSLKLSCAASGFTFNT |
| | YAMNWVRQAPGKGLEWVGRIRSKYNNYATYYADSV |
| | KDRFTISRDDSQNILYLQMNNLRTEDTAVYYCVRHGN |
| | FGNSYVSWFAYWGQGTLVTVSSGGGGSGGGGSGGG |
| | GSAQAFVTQEPSLTVSPGGTVTLTCGSSTGAVTTSNYA |
| | NWVQQKPGQAPRGLIGGTNKRAPGVPARFSGSLIGDK |
| | AALTITGAQPEDEAEYYCALWYSNLWVFGGGTKLTV |
| | LEPKSSDKTHTCPPCPAPEAAGAPSVFLFPPKPKDTLMI |
| | SRTPEVTCVVVDVSHEDPEVKFNWYVDGVEVHNAKT |
| | KPREEQYNSTYRVVSVLTVLHQDWLNGKEYKCKVSN |
| | KALPAPIEKTISKAKGQPREPQVYTLPPCRDELTKNQV |
| | SLWCLVKGFYPSDIA VEWESNGQPENNYKTTPPVLDS |
| | DGSFFLYSKLTVDKSRWQQGNVFSCSVMHEALHNRF |
| | TQKSLSLSPG (SEQ ID NO: 77) |
| huA14.QF_G1AAA_hole | QVQLVQSGAEVKKPGASVKVSCKASGYTFTDYYMN |
| | WVRQAPGQGLEWMGVINPYNGVPQYSQKFKDRVTM |
| | TRDTSISTAYMELSRLRSDDTVVYYCVRAREFFFYFDV |
| | WGQGTLVTVSSASTKGPSVFPLAPSSKSTSGGTAALGC |
| | LVKDYFPEPVTVSWNSGALTSGVHTFPAVLQSSGLYS |
| | LSSVVTVPSSSLGTQTYICNVNHKPSNTKVDKKVEPKS |
| | CDKTHTCPPCPAPEAAGAPSVFLFPPKPKDTLMISRTPE |
| | VTCVVVDVSHEDPEVKFNWYVDGVEVHNAKTKPREE |
| | QYNSTYRVVSVLTVLHQDWLNGKEYKCKVSNKALPA |
| | PIEKTISKAKGQPREPQVCTLPPSRDELTKNQVSLSCAV |
| | KGFYPSDIAVEWESNGQPENNYKTTPPVLDSDGSFFLV |
| | SKLTVDKSRWQQGNVFSCSVMHEALHNHYTQKSLSL |
| | SPG (SEQ ID NO: 78) |
| huA14.QF_LC | DIQMTQSPSSLSASVGDRVTITCSASSSINYMHWYQQK |
| | PGKAPKLLIYDTSKLPSGVPSRFSGSGSGTDFTLTISSLQ |
| | PEDFATYYCQQWDSNPPTFGGGTKVEIKRTVAAPSVFI |
| | FPPSDEQLKSGTASVVCLLNNFYPREAKVQWKVDNAL |
| | QSGNSQESVTEQDSKDSTYSLSSTLTLSKADYEKHKV |
| | YACEVTHQGLSSPVTKSFNRGEC (SEQ ID NO: 79) |
| hu2A5_1B10_G1AAA_knob_RF | QVQLVQSGAEVKKPGASVKVSCKASGYTLTDYYMN |
| | WVRQAPGQGLEWMGIINPYNGASTYNQNFRGRVTMT |
| | RDTSTSTVYMELSSLRSEDTAVYYCARPPYDGYFEGL |
| | EYWGQGTLVTVSSASTKGPSVFPLAPSSKSTSGGTAAL |
| | GCLVKDYFPEPVTVSWNSGALTSGVHTFPAVLQSSGL |
| | YSLSSVVTVPSSSLGTQTYICNVNHKPSNTKVDKKVEP |
| | KSCDKTHTEVKLLESGGGLVQPGGSLKLSCAASGFTF |
| | NTYAMNWVRQAPGKGLEWVGRIRSKANNYATYYAD |
| | SVKGRFTISRDDSKNTLYLQMNNLKAEDTAVYYCVR |
| | HGNFGNSYVSWFAYWGQGTLVTVSSGGGGSGGGGS |
| | GGGGSAEAVVTQEPSLTVSPGGTVTLTCGSSTGAVTTS |
| | NYANWVQQKPGQAPRGLIGGTNKRAPGTPARFSGSLL |
| | GDKAALTITGAQPEDEAEYYCALWYSNLWVFGGGTK |
| | LTVLEPKSSDKTHTCPPCPAPEAAGAPSVFLFPPKPKDT |
| | LMISRTPEVTCVVVDVSHEDPEVKFNWYVDGVEVHN |
| | AKTKPREEQYNSTYRVVSVLTVLHQDWLNGKEYKCK |
| | VSNKALPAPIEKTISKAKGQPREPQVYTLPPCRDELTK |
| | NQVSLWCLVKGFYPSDIAVEWESNGQPENNYKTTPPV |
| | LDSDGSFFLYSKLTVDKSRWQQGNVFSCSVMHEALH |
| | NRFTQKSLSLSPG (SEQ ID NO: 80) |
| hu2A5_1B4_G1AAA_knob_RF | QVQLVQSGAEVKKPGASVKVSCKASGYTLTDYYMN |
| | WVRQAPGQGLEWMGIINPYNGASTYNQNFRGRVTMT |
| | RDTSTSTVYMELSSLRSEDTAVYYCARPPYDGYFEGL |
| | EYWGQGTLVTVSSASTKGPSVFPLAPSSKSTSGGTAAL |
| | GCLVKDYFPEPVTVSWNSGALTSGVHTFPAVLQSSGL |
| | YSLSSVVTVPSSSLGTQTYICNVNHKPSNTKVDKKVEP |
| | KSCDKTHTEVKLVESGGGLVQPGGSLKLSCAASGFTF |
| | NTYAMNWVRQAPGKGLEWVGRIRSKYNNYATYYAD |
| | SVKDRFTISRDDSQNILYLQMNNLRTEDTAVYYCVRH |
| | GNFGNSYVSWFAYWGQGTLVTVSSGGGGSGGGGSG |
| | GGGSAQAFVTQEPSLTVSPGGTVTLTCGSSTGAVTTSN |
| | YANWVQQKPGQAPRGLIGGTNKRAPGVPARFSGSLIG |
| | DKAALTITGAQPEDEAEYYCALWYSNLWVFGGGTKL |
| | TVLEPKSSDKTHTCPPCPAPEAAGAPSVFLFPPKPKDTL |
| | MISRTPEVTCVVVDVSHEDPEVKFNWYVDGVEVHNA |
| | KTKPREEQYNSTYRVVSVLTVLHQDWLNGKEYKCKV |
| | SNKALPAPIEKTISKAKGQPREPQVYTLPPCRDELTKN |
| | QVSLWCLVKGFYPSDIAVEWESNGQPENNYKTTPPVL |
| | DSDGSFFLYSKLTVDKSRWQQGNVFSCSVMHEALHN |
| | RFTQKSLSLSPG (SEQ ID NO: 81) |
| hu2A5_G1AAA_hole | QVQLVQSGAEVKKPGASVKVSCKASGYTLTDYYMN |
| | WVRQAPGQGLEWMGIINPYNGASTYNQNFRGRVTMT |
| | RDTSTSTVYMELSSLRSEDTAVYYCARPPYDGYFEGL |
| | EYWGQGTLVTVSSASTKGPSVFPLAPSSKSTSGGTAAL |
| | GCLVKDYFPEPVTVSWNSGALTSGVHTFPAVLQSSGL |
| | YSLSSVVTVPSSSLGTQTYICNVNHKPSNTKVDKKVEP |
| | KSCDKTHTCPPCPAPEAAGAPSVFLFPPKPKDTLMISR |
| | TPEVTCVVVDVSHEDPEVKFNWYVDGVEVHNAKTKP |
| | REEQYNSTYRVVSVLTVLHQDWLNGKEYKCKVSNKA |
| | LPAPIEKTISKAKGQPREPQVCTLPPSRDELTKNQVSLS |
| | CAVKGFYPSDIAVEWESNGQPENNYKTTPPVLDSDGS |
| | FFLVSKLTVDKSRWQQGNVFSCSVMHEALHNHYTQK |
| | SLSLSPG (SEQ ID NO: 82) |
| hu2A5_LC | DIQMTQSPSSLSASVGDRVTITCKASQDVSTAVAWYQ |
| | QKPGKAPKLLIYSASYRYTGVPSRFSGSGSGTDFTLTIS |
| | SLQPEDFATYYCQQHYFTPRTFGGGTKVEIKRTVAAPS |
| | VFIFPPSDEQLKSGTASVVCLLNNFYPREAKVQWKVD |
| | NALQSGNSQESVTEQDSKDSTYSLSSTLTLSKADYEKH |
| | KVYACEVTHQGLSSPVTKSFNRGEC (SEQ ID NO: 83) |

In one preferred embodiment, a multispecific antibody binds to CLEC12A and CD38, and comprises a first light chain subunit comprising SEQ ID NO: 79; a first heavy chain subunit comprising SEQ ID NO: 76; a second light chain subunit comprising SEQ ID NO: 79; and a second heavy chain subunit comprising SEQ ID NO: 78.

In one preferred embodiment, a multispecific antibody binds to CLEC12A and CD38, and comprises a first light chain subunit comprising SEQ ID NO: 79; a first heavy chain subunit comprising SEQ ID NO: 77; a second light chain subunit comprising SEQ ID NO: 79; and a second heavy chain subunit comprising SEQ ID NO: 78.

In one preferred embodiment, a multispecific antibody binds to CLEC12A and CD38, and comprises a first light chain subunit comprising SEQ ID NO: 83; a first heavy chain subunit comprising SEQ ID NO: 80; a second light chain subunit comprising SEQ ID NO: 83; and a second heavy chain subunit comprising SEQ ID NO: 82.

In one preferred embodiment, a multispecific antibody binds to CLEC12A and CD38, and comprises a first light chain subunit comprising SEQ ID NO: 83; a first heavy chain subunit comprising SEQ ID NO: 81; a second light chain subunit comprising SEQ ID NO: 83; and a second heavy chain subunit comprising SEQ ID NO: 82.

Various formats of multi-specific antibodies are within the ambit of the invention, including, without limitation, single chain polypeptides, two chain polypeptides, three chain polypeptides, four chain polypeptides, and multiples thereof. The multi-specific antibodies herein specifically include T-cell multi-specific (e.g., bispecific) antibodies binding to CLEC12A and CD38 (anti-CLEC12A x anti-CD3& antibodies). Such antibodies induce potent T-cell mediated killing of cells expressing CLEC12A.

### Preparation of Anti-CLEC12A Antibodies

The antibodies of the present invention can be prepared by methods known in the art, such as recombinant DNA technology, by expression of the encoding nucleic acid in a suitable eukaryotic or prokaryotic host, including, for example, mammalian cells (e.g., CHO cells), E. coli or yeast.

Antibodies binding to non-overlapping epitopes on a CLEC12A protein can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc).

Typically, an antibody “competes” with a reference antibody if it causes about 15-100% reduction in the binding of the reference antibody to the target antigen, as determined by standard techniques, such as by the competition binding assays described above. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Pharmaceutical Compositions, Uses and Methods of Treatment

It is another aspect of the present invention to provide pharmaceutical compositions comprising one or more antibodies of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises an antibody that binds to CLEC12A. In another embodiment, a pharmaceutical composition comprises a multi-specific (including bispecific) antibody with binding specificity for two or more non-overlapping epitopes on a CLEC12A protein. In a preferred embodiment, a pharmaceutical composition comprises a multi-specific (including bispecific) antibody that binds to CLEC12A and a binding target on an effector cell (e.g., a binding target on a T-cell, such as, e.g., a CD3& protein on a T-cell).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen.

### Methods of Use

The anti-CLEC12A antibodies and pharmaceutical compositions described herein can be used for the treatment of diseases and conditions characterized by the expression of CLEC12A, including, without limitation, the conditions and diseases described further herein.

CLEC12A, also known as C-type Lectin Domain Family 12 Member A, DCAL-2, CLL-1, MICL and CD371, is a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function.

RNA expression analysis from TCGA databases shows that CLEC12A is highly expressed and specific to acute myeloid leukemia. Therapeutic development of antibodies targeting CLEC12A may therefore be efficacious in treating AML patients, potentially including a significant number of AML patients who are refractory to current standard of care treatments.

In one aspect, the anti-CLEC12A antibodies and pharmaceutical compositions herein can be used to treat disorders characterized by the expression of CLEC12A, including, without limitation, hematologic malignancies that begin in blood-forming tissues, including, but not limited to, bone marrow and/or cells of the immune system. In one preferred embodiment, the anti-CLEC12A antibodies and pharmaceutical compositions herein are used to treat AML. In one preferred embodiment, the AML is refractory AML, wherein the patient receiving treatment has previously received another therapy (e.g., a chemotherapy regimen) and has not achieved remission of the AML.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration.

Therapeutic entities of the present invention are usually administered on multiple occasions. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other ablative agent dissolved in a pharmaceutically acceptable carrier.

Also within the scope of the invention are kits comprising the active agents and formulations thereof, of the invention and instructions for use. The kit can further contain at least one additional reagent. Kits typically include a label indicating the intended use of the contents of the kit. The term “label” as used herein includes any writing, or recorded material supplied on or with a kit, or which otherwise accompanies a kit.

The invention now being fully described, it will be apparent to one of ordinary skill in the art that various changes and modifications can be made without departing from the spirit or scope of the invention.

### EXAMPLES

### Example 1: Monovalent Binding Kinetics of Humanized Anti-CLEC12A Antibodies

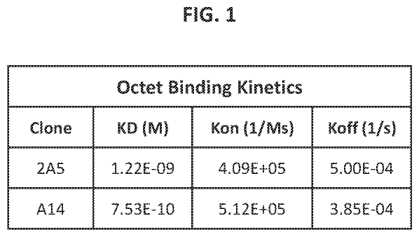

Purified antibodies were characterized for affinity to the antigen using Octet Red96 by loading purified antibodies onto anti-human Heavy Chain (AHC) capture sensors and measuring rates of association and dissociation of histidine tagged recombinant human CLEC12A protein. The results are shown in FIG. 1.

### Example 2: Binding Analysis of Humanized Anti-CLEC12A Antibodies Against CLEC12A-Expressing Cell Lines and Primary Cells

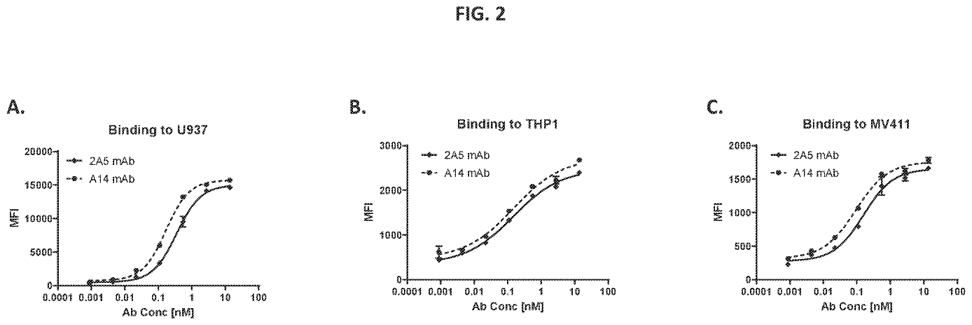

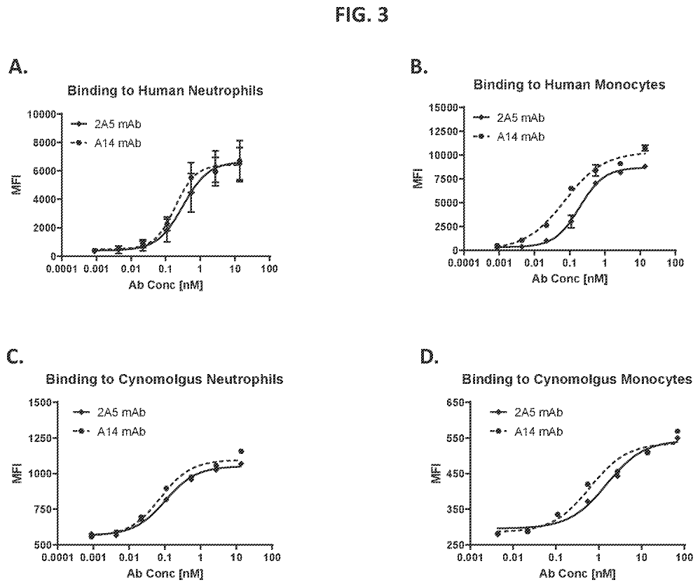

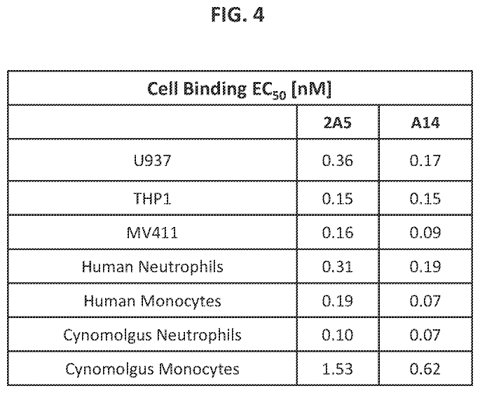

Humanized anti-CLEC12A antibodies were tested for their binding to cells expressing CLEC12A. Three acute myeloid leukemia cell lines expressing different levels of CLEC12A were tested, along with human and cynomolgus monocytes and neutrophils that were isolated from PBMCs. Cells were washed with and resuspended in FACS buffer. Cells were plated onto 96 well plates at 1E5 cells/well. Cells were stained with titrated anti-CLEC12A antibodies on ice for 30 minutes followed by washing and secondary staining with 1:500 diluted goat anti-human IgG-AF647. Cells were analyzed by flow cytometry following final washes and addition of 7-AAD. The geometric means of fluorescence intensity were graphed against the concentration of antibodies to generate a dose response curve. FIG. 2, panels A-C, show binding curves to the indicated AML cell lines (U937, THP1 and MV411). FIG. 3, panels A-D, show binding curves to human and cynomolgus neutrophils and monocytes. FIG. 4 summarizes the EC₅₀ values of cell binding.

### Example 3: Binding of Bispecific CLEC12A×CD3 Bispecific Antibodies to Acute Myeloid Leukemia Cells Expressing CLEC12A and to T-Cells

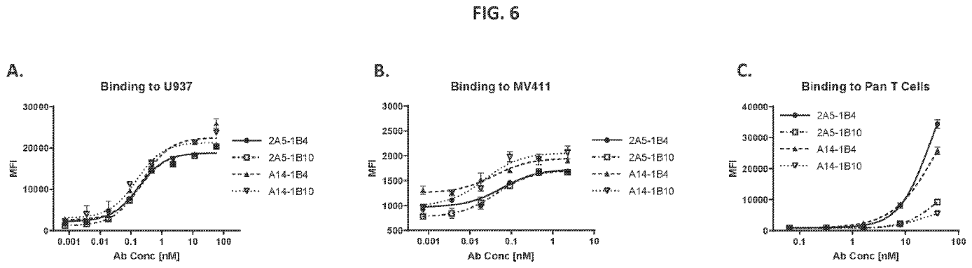

CLEC12A×CD3 bispecific antibodies were assessed for their binding to AML cell lines expressing CLEC12A and T-cells expressing CD3. Cells were plated in a 96-well V-bottom plate at a density of 1×10⁵. Serially diluted antibodies were added to the cells and incubated for 30 min on ice. Following 2 washes with FACS buffer, Alexa Fluor 647 labeled anti-human Fc secondary antibodies were added and incubated for 20 min on ice. Following 2 more washes with FACS buffer, cells were resuspended in FACS buffer containing 7AAD cell viability dye and analyzed on a flow cytometer. The geometric means of fluorescence intensity were graphed against the concentration of antibodies to generate a dose response curve. The results are shown in FIG. 6, panels A-C.

### Example 4: Binding of CLEC12A×CD3 Bispecific Antibodies to CLEC12 and CD3 on a Sandwich ELISA

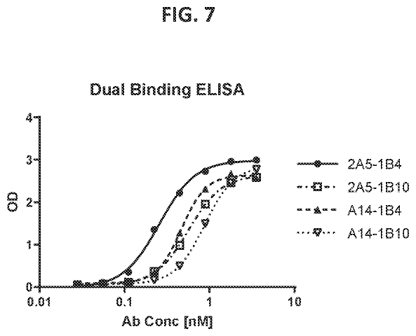

A sandwich ELISA was used to assess the concurrent binding to CLEC12A and CD3. Recombinant His-tagged CD3 protein was coated onto a 96-well plate overnight at room temperature with shaking. After washing the plate with PBS containing 0.05% Tween-20, the plate was blocked with 2% BSA for 60 min and antibodies were added and incubated for 60 min at room temperature with shaking. After washing, biotinylated recombinant CLEC12A protein was added to the plate and incubated for 60 min at room temperature. The plate was washed and HRP (horseradish peroxidase) conjugated streptavidin was added and incubated for 30 min at room temperature. After washing, TMB (3,3′,5,5′-tetramethylbenzidine) substrate was added and incubated for 5 to 10 min to develop color, after which 0.16 M sulfuric acid was added to stop the reaction. Absorbance was read on a plate reader and plotted against antibody concentrations to generate a dose response curve. The results are shown in FIG. 7.

### Example 5: Proliferation of CD8+ T-Cells Mediated by CLEC12A×CD3 Bispecific Antibodies in the Presence of Autologous Monocytes Expressing CLEC12A

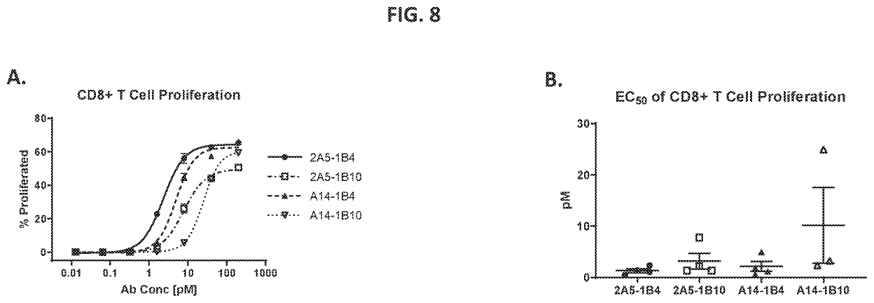

CLEC12A×CD3 bispecific antibodies were validated for their biological activity in stimulating T-cells. First, the proliferation of T-cells in the presence of autologous monocytes was tested. T-cells and CD14+ monocytes were isolated from PBMCs of healthy donors. Monocytes were seeded at 1×10⁴ cells per well in a U-bottom 96-well plate in RPMI1640 medium supplemented with 10% heat-inactivated human serum. T-cells were labeled with CellTrace Violet dye and 5×10⁴ cells were added to wells containing monocytes. CLEC12A×CD3 bispecific antibodies were then added, and cells were incubated for 5 days. On the day of analysis, cells were harvested and stained for CD4, CD8 and cell viability. Samples were analyzed on a flow cytometer. Dilution of CellTrace Violet dye was gated and reported as % proliferation, then plotted against antibody concentrations. The results are shown in FIG. 8, panels A-B.

### Example 6: Cytokine Release from T-Cells Mediated by CLEC12A×CD3 Bispecific Antibodies in the Presence of U937 Expressing CLEC12A

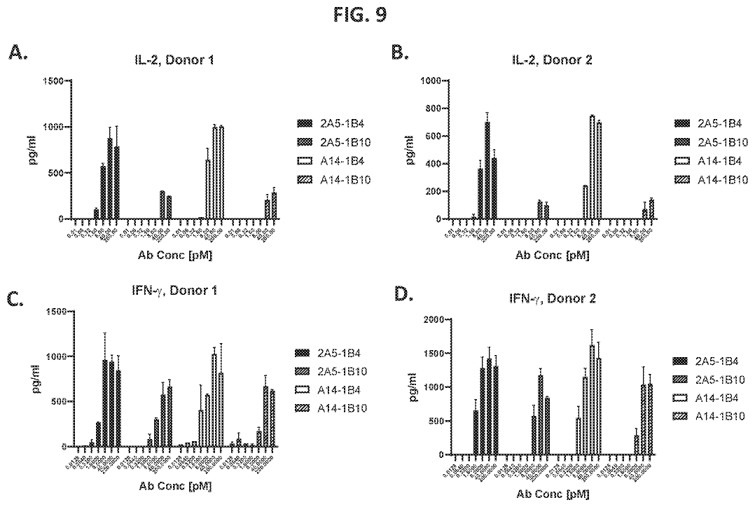

CLEC12A×CD3 bispecific antibodies were tested for their stimulation of cytokine release from T-cells. Human PBMCs were isolated and incubated with CLEC12A×CD3 bispecific antibodies and U937 cells expressing CLEC12A. Supernatant was collected after 48 h for IL-2 and IFNγ release detection was conducted using commercially available ELISA kits. The results are shown in FIG. 9, panels A-D.

### Example 7: CD8+ T-Cell Activation and Cell Killing Activity

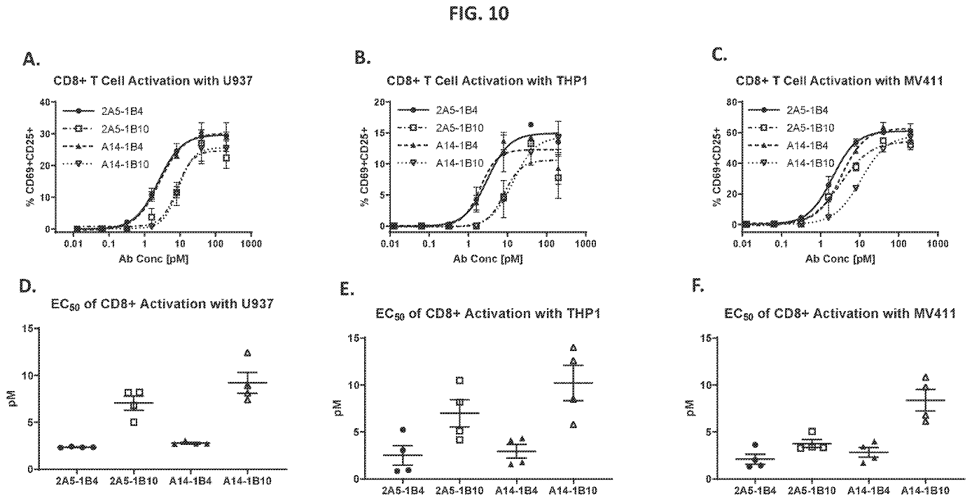

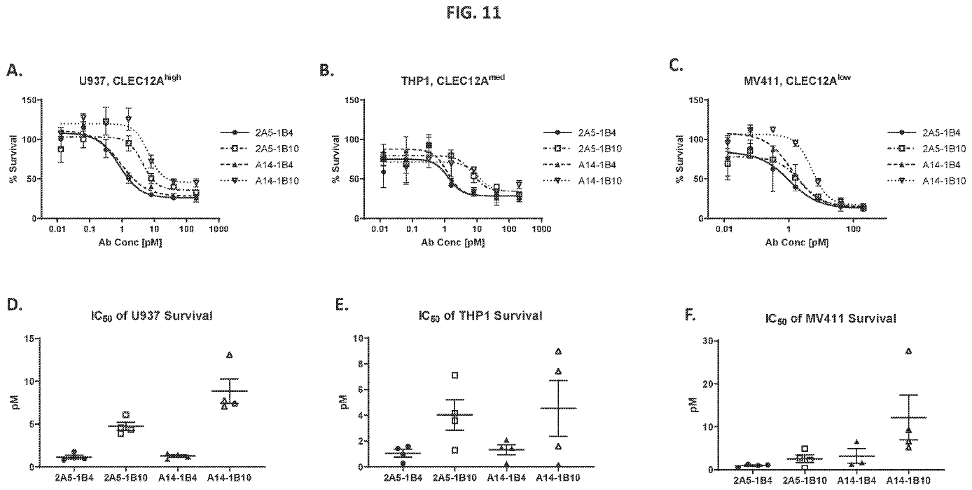

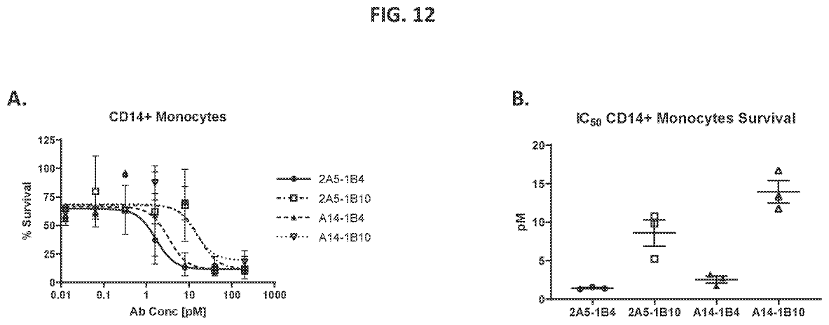

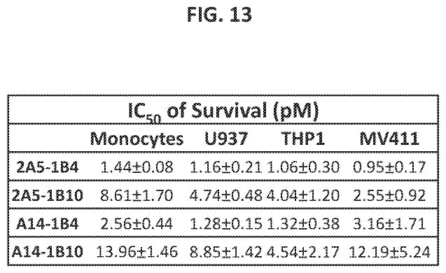

CLEC12A×CD3 bispecific antibodies were tested for their potency in activating T-cells and mediating killing of AML target cells and autologous monocytes. CD8+ T-cells were isolated from PBMCs and rested overnight in RPMI1640 medium supplemented with 20 U/mL IL-2. U937, THP1, MV411 AML cell lines and autologous CD14+ monocytes expressing CLEC12A were labeled with CellTrace Violet and co-cultured with the isolated CD8+ T-cells in the presence of titrated CLEC12A×CD3 bispecific antibodies for 24 hours. Cells were harvested the next day and stained for CD8, CD25 and CD69 and with 7-AAD cell viability dye. Samples were analyzed by flow cytometry after the addition of counting beads. For T-cell activation, the percentage of CD8+ labeled cells expressing CD25 and CD69 was plotted against the concentration of antibodies. For cytotoxicity, the number of live AML cells was counted and reported as a percentage of AML cells in the control well without T-cells or antibodies. FIG. 10, panels A-F, show the activation of CD8+ T-cells. FIG. 11, panels A-F, show the killing of AML cell lines. FIG. 12, panels A-B, show the cytotoxicity on autologous monocytes. FIG. 13 summarizes the IC₅₀ of survival for AML cell lines and autologous monocytes.

### Example 8: Anti-Tumor Efficacy of CLEC12A×CD3 Bispecific Antibodies in a U937/Human PBMC Co-Graft Model in NSG Mice

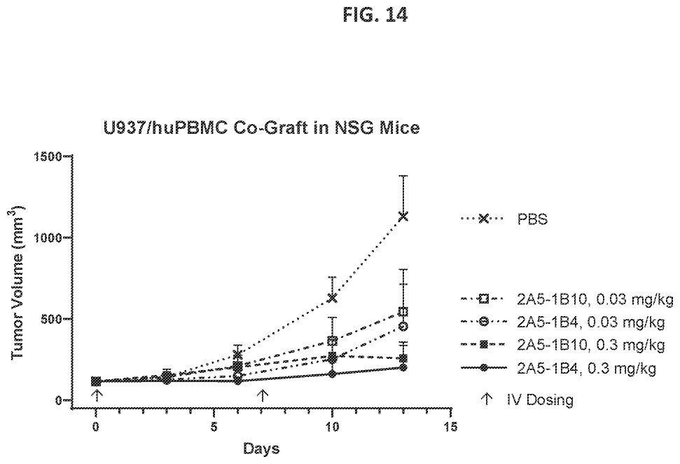

CLEC12A×CD3 bispecific antibodies were tested for their ability to suppress the growth of CLEC12A positive U937 human cancer cells in vivo. In a co-graft model, U937 cells were co-implanted with human PBMCs in the flanks of NSG mice and let grow until around 100 mm3, after which CLEC12A×CD3 bispecific antibodies were dosed intravenously weekly for a total of 2 doses. Antibody construct 2A5-1B4 was more potent than 2A51-1B10, at both the low and high dose. The results are shown in FIG. 14.

### Example 9: Anti-Tumor Efficacy of CLEC12A×CD3 Bispecific Antibodies in a U937/Human PBMC Reconstitution Model in NSG Mice

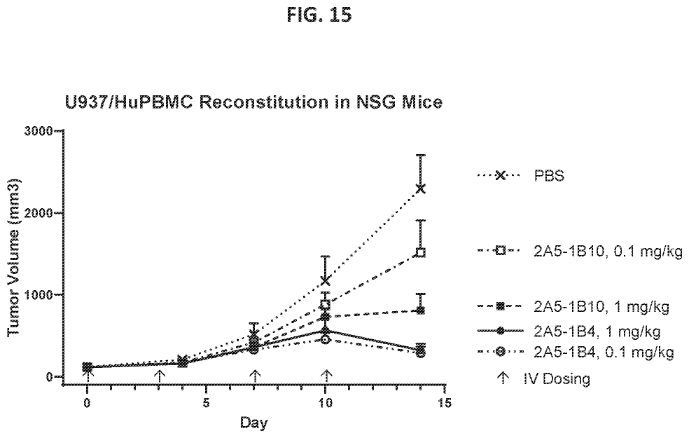

In a second model, U937 cells were first implanted in the flanks of NSG mice and let grow until around 100 mm3, after which human PBMCs were grafted intraperitoneally. Intravenous injection of CLEC12A×CD3 bispecific antibodies started after 48 hours, twice weekly, for a total of 4 doses. Consistent with results obtained from the co-graft model, antibody construct 2A5-1B4 was more effective in suppressing the growth of U937 cells. The results are shown in FIG. 15.

### Example 10: Monovalent Binding Kinetics of CLEC12A×CD3 Bispecific Antibody to CLEC12A and CD3 Epsilon

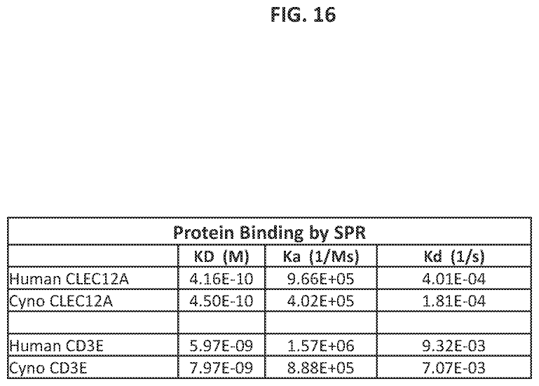

Binding kinetics of CLEC12A×CD3 bispecific antibody 2A5-1B4 were characterized using SPR. Specifically, a capture coupling method was used. Briefly, anti-human IgG (Fc) antibodies were first immobilized on the reference and experimental channels of a CM5 chip using amino coupling, followed by capturing of the CLEC12A×CD3 bispecific antibody. Serially diluted recombinant human CLEC12A or CD3 epsilon proteins were then flowed through the surface of the two channels for association and dissociation. Biacore 8K Evaluation software was used to analyze and evaluate the results. The average value of 3 technical replicates was reported as the assay result. Results are provided in FIG. 16. The results showed higher binding affinity for CLEC12A than CD3 epsilon. In addition, the binding affinity is similar between human and cynomolgus proteins.

### Example 11: Anti-Tumor Efficacy of CLEC12A×CD3 Bispecific Antibodies in an Orthotopic HL-60-Luc2 AML Model

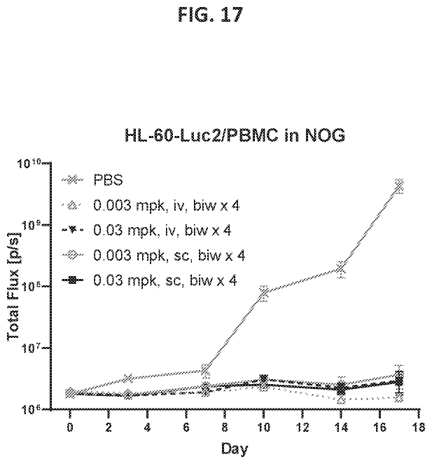

In an orthotopic AML model using HL-60-Luc2 expressing luciferase for in vivo imaging, CLEC12A×CD3 bispecific antibody 2A5-1B4 was dosed biweekly via intravenous (iv) or subcutaneous (sc) injection for a total of 8 doses. Results are provided in FIG. 17. Antibody 2A5-1B4 suppressed tumor growth at the lowest dose tested of 0.003 mpk using either route of administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antibody that binds to CLEC12A, comprising a first binding unit comprising:
a heavy chain variable region comprising:
(a) a CDRH1 sequence of any one of SEQ ID NOs: 1-5; and/or
(b) a CDRH2 sequence of any one of SEQ ID NOs: 6-14; and/or
(c) a CDRH3 sequence of any one of SEQ ID NOs: 15-22; and
a light chain variable region comprising:
(d) a CDRL1 sequence of any one of SEQ ID NOs: 23-27; and/or
(e) a CDRL2 sequence of any one of SEQ ID NOs: 28-33; and/or
(f) a CDRL3 sequence of any one of SEQ ID NOs: 34-40.

2. The antibody of claim 1, wherein the CDRH1, CDRH2 and CDRH3 sequences of the first binding unit are present in a human VH framework.

3. The antibody of claim 1 or 2, wherein the CDRL1, CDRL2 and CDRL3 sequences of the first binding unit are present in a human VL framework.

4. The antibody of any one of claims 1-3, wherein the first binding unit comprises:
a heavy chain variable region comprising:
(a) a CDRH1 sequence of any one of SEQ ID NOs: 1-5; and
(b) a CDRH2 sequence of any one of SEQ ID NOs: 6-14; and
(c) a CDRH3 sequence of any one of SEQ ID NOs: 15-22.

5. The antibody of claim 4, wherein the first binding unit comprises:
a heavy chain variable region comprising:
(a) a CDRH1 sequence of SEQ ID NO: 1, a CDRH2 sequence of SEQ ID NO: 6, and a CDRH3 sequence of SEQ ID NO: 15; or
(b) a CDRH1 sequence of SEQ ID NO: 1, a CDRH2 sequence of SEQ ID NO: 7, and a CDRH3 sequence of SEQ ID NO: 16; or
(c) a CDRH1 sequence of SEQ ID NO: 1, a CDRH2 sequence of SEQ ID NO: 8, and a CDRH3 sequence of SEQ ID NO: 17; or
(d) a CDRH1 sequence of SEQ ID NO: 1, a CDRH2 sequence of SEQ ID NO: 9, and a CDRH3 sequence of SEQ ID NO: 18; or
(e) a CDRH1 sequence of SEQ ID NO: 2, a CDRH2 sequence of SEQ ID NO: 10, and a CDRH3 sequence of SEQ ID NO: 19; or
(f) a CDRH1 sequence of SEQ ID NO: 3, a CDRH2 sequence of SEQ ID NO: 11, and a CDRH3 sequence of SEQ ID NO: 20; or
(g) a CDRH1 sequence of SEQ ID NO: 1, a CDRH2 sequence of SEQ ID NO: 12, and a CDRH3 sequence of SEQ ID NO: 21; or
(h) a CDRH1 sequence of SEQ ID NO: 4, a CDRH2 sequence of SEQ ID NO: 13, and a CDRH3 sequence of SEQ ID NO: 22; or
(i) a CDRH1 sequence of SEQ ID NO: 5, a CDRH2 sequence of SEQ ID NO: 14, and a CDRH3 sequence of SEQ ID NO: 22.

6. The antibody of any one of claims 1-5, wherein the first binding unit comprises:
a light chain variable region comprising:
(a) a CDRL1 sequence of any one of SEQ ID NOs: 23-27; and
(b) a CDRL2 sequence of any one of SEQ ID NOs: 28-33; and
(c) a CDRL3 sequence of any one of SEQ ID NOs: 34-40.

7. The antibody of claim 6, wherein the first binding unit comprises:
a light chain variable region comprising:
(a) a CDRL1 sequence of SEQ ID NO: 23, a CDRL2 sequence of SEQ ID NO: 28, and a CDRL3 sequence of SEQ ID NO: 34; or
(b) a CDRL1 sequence of SEQ ID NO: 24, a CDRL2 sequence of SEQ ID NO: 29, and a CDRL3 sequence of SEQ ID NO: 35; or
(c) a CDRL1 sequence of SEQ ID NO: 25, a CDRL2 sequence of SEQ ID NO: 30, and a CDRL3 sequence of SEQ ID NO: 36; or
(d) a CDRL1 sequence of SEQ ID NO: 26, a CDRL2 sequence of SEQ ID NO: 31, and a CDRL3 sequence of SEQ ID NO: 37; or
(e) a CDRL1 sequence of SEQ ID NO: 25, a CDRL2 sequence of SEQ ID NO: 30, and a CDRL3 sequence of SEQ ID NO: 38; or
(f) a CDRL1 sequence of SEQ ID NO: 27, a CDRL2 sequence of SEQ ID NO: 32, and a CDRL3 sequence of SEQ ID NO: 39; or
(g) a CDRL1 sequence of SEQ ID NO: 24, a CDRL2 sequence of SEQ ID NO: 33, and a CDRL3 sequence of SEQ ID NO: 40.

8. The antibody of any one of claims 1-7, wherein the first binding unit comprises:
(a) a heavy chain variable region comprising:
a CDRH1 sequence of SEQ ID NO: 1, a CDRH2 sequence of SEQ ID NO: 6, and a CDRH3 sequence of SEQ ID NO: 15; and
a light chain variable region comprising:
a CDRL1 sequence of SEQ ID NO: 23, a CDRL2 sequence of SEQ ID NO: 28, and a CDRL3 sequence of SEQ ID NO: 34; or
(b) a heavy chain variable region comprising:
a CDRH1 sequence of SEQ ID NO: 1, a CDRH2 sequence of SEQ ID NO: 7, and a CDRH3 sequence of SEQ ID NO: 16; and
a light chain variable region comprising:
a CDRL1 sequence of SEQ ID NO: 23, a CDRL2 sequence of SEQ ID NO: 28, and a CDRL3 sequence of SEQ ID NO: 34; or
(c) a heavy chain variable region comprising:
a CDRH1 sequence of SEQ ID NO: 1, a CDRH2 sequence of SEQ ID NO: 8, and a CDRH3 sequence of SEQ ID NO: 17; and
a light chain variable region comprising:
a CDRL1 sequence of SEQ ID NO: 24, a CDRL2 sequence of SEQ ID NO: 29, and a CDRL3 sequence of SEQ ID NO: 35; or
(d) a heavy chain variable region comprising:
a CDRH1 sequence of SEQ ID NO: 1, a CDRH2 sequence of SEQ ID NO: 9, and a CDRH3 sequence of SEQ ID NO: 18; and
a light chain variable region comprising:
a CDRL1 sequence of SEQ ID NO: 25, a CDRL2 sequence of SEQ ID NO: 30, and a CDRL3 sequence of SEQ ID NO: 36; or
(e) a heavy chain variable region comprising:
a CDRH1 sequence of SEQ ID NO: 2, a CDRH2 sequence of SEQ ID NO: 10, and a CDRH3 sequence of SEQ ID NO: 19; and
a light chain variable region comprising:
a CDRL1 sequence of SEQ ID NO: 26, a CDRL2 sequence of SEQ ID NO: 31, and a CDRL3 sequence of SEQ ID NO: 37; or
(f) a heavy chain variable region comprising:
a CDRH1 sequence of SEQ ID NO: 1, a CDRH2 sequence of SEQ ID NO: 6, and a CDRH3 sequence of SEQ ID NO: 15; and
a light chain variable region comprising:
a CDRL1 sequence of SEQ ID NO: 23, a CDRL2 sequence of SEQ ID NO: 28, and a CDRL3 sequence of SEQ ID NO: 34; or
(g) a heavy chain variable region comprising:
a CDRH1 sequence of SEQ ID NO: 3, a CDRH2 sequence of SEQ ID NO: 11, and a CDRH3 sequence of SEQ ID NO: 20; and
a light chain variable region comprising:
a CDRL1 sequence of SEQ ID NO: 25, a CDRL2 sequence of SEQ ID NO: 30, and a CDRL3 sequence of SEQ ID NO: 38; or
(h) a heavy chain variable region comprising:
a CDRH1 sequence of SEQ ID NO: 1, a CDRH2 sequence of SEQ ID NO: 12, and a CDRH3 sequence of SEQ ID NO: 21; and
a light chain variable region comprising:
a CDRL1 sequence of SEQ ID NO: 27, a CDRL2 sequence of SEQ ID NO: 32, and a CDRL3 sequence of SEQ ID NO: 39; or
(i) a heavy chain variable region comprising:
a CDRH1 sequence of SEQ ID NO: 4, a CDRH2 sequence of SEQ ID NO: 13, and a CDRH3 sequence of SEQ ID NO: 22; and
a light chain variable region comprising:
a CDRL1 sequence of SEQ ID NO: 24, a CDRL2 sequence of SEQ ID NO: 33, and a CDRL3 sequence of SEQ ID NO: 40; or
(j) a heavy chain variable region comprising:
a CDRH1 sequence of SEQ ID NO: 5, a CDRH2 sequence of SEQ ID NO: 14, and a CDRH3 sequence of SEQ ID NO: 22; and
a light chain variable region comprising:
a CDRL1 sequence of SEQ ID NO: 24, a CDRL2 sequence of SEQ ID NO: 33, and a CDRL3 sequence of SEQ ID NO: 40.

9. The antibody of claim 8, wherein the first binding unit comprises a heavy chain variable region sequence having at least 95% identity to any one of SEQ ID NOs: 41-52.

10. The antibody of claim 8, wherein the first binding unit comprises a heavy chain variable region sequence comprising any one of SEQ ID NOs: 41-52.

11. The antibody of any one of claims 8-10, wherein the first binding unit comprises a light chain variable region sequence having at least 95% identity to any one of SEQ ID NOs: 53-64.

12. The antibody of any one of claims 8-10, wherein the first binding unit comprises a light chain variable region sequence comprising any one of SEQ ID NOs: 53-64.

13. The antibody of any one of claims 8-12, wherein the first binding unit comprises:
(a) a heavy chain variable region sequence of SEQ ID NO: 41 and a light chain variable region sequence of SEQ ID NO: 53; or
(b) a heavy chain variable region sequence of SEQ ID NO: 42 and a light chain variable region sequence of SEQ ID NO: 54; or
(c) a heavy chain variable region sequence of SEQ ID NO: 43 and a light chain variable region sequence of SEQ ID NO: 55; or
(d) a heavy chain variable region sequence of SEQ ID NO: 44 and a light chain variable region sequence of SEQ ID NO: 56; or
(e) a heavy chain variable region sequence of SEQ ID NO: 45 and a light chain variable region sequence of SEQ ID NO: 57; or
(f) a heavy chain variable region sequence of SEQ ID NO: 46 and a light chain variable region sequence of SEQ ID NO: 58; or
(g) a heavy chain variable region sequence of SEQ ID NO: 47 and a light chain variable region sequence of SEQ ID NO: 59; or
(h) a heavy chain variable region sequence of SEQ ID NO: 48 and a light chain variable region sequence of SEQ ID NO: 60; or
(i) a heavy chain variable region sequence of SEQ ID NO: 49 and a light chain variable region sequence of SEQ ID NO: 61; or
(j) a heavy chain variable region sequence of SEQ ID NO: 50 and a light chain variable region sequence of SEQ ID NO: 62; or
(k) a heavy chain variable region sequence of SEQ ID NO: 51 and a light chain variable region sequence of SEQ ID NO: 63; or
(l) a heavy chain variable region sequence of SEQ ID NO: 52 and a light chain variable region sequence of SEQ ID NO: 64.

14. The antibody of any one of claims 1-13, further comprising a heavy chain constant region.

15. The antibody of claim 14, wherein the heavy chain constant region comprises a hinge region, a CHI region, a CH2 region, and/or a CH3 region.

16. The antibody of any one of claims 14-15, wherein the heavy chain constant region comprises one or more knob-in-hole (KiH) mutations.

17. The antibody of any one of claims 14-16, wherein the heavy chain constant region comprises one or more silencing mutations.

18. The antibody of any one of claims 14-17, wherein the heavy chain constant region comprises one or more protein A binding mutations.

19. The antibody of claim 18, wherein the one or more protein A binding mutations comprise an H435R mutation, a Y436F mutation, or both an H435R and a Y436F mutation.

20. The antibody of any one of claims 1-19, further comprising a light chain constant region.

21. The antibody of claim 20, wherein the light chain constant region comprises a CL region.

22. The antibody of any one of claims 1-21, which is monospecific.

23. The antibody of any one of claims 1-21, which is multispecific.

24. The antibody of claim 23, which is bispecific.

25. The antibody of claim 23 or 24, further comprising a second binding unit that binds to CD38.

26. The antibody of claim 25, wherein the second binding unit comprises:
a heavy chain variable region comprising:
(a) a CDRH1 sequence of SEQ ID NO: 65; and/or
(b) a CDRH2 sequence of any one of SEQ ID NOs: 66-67; and/or
(c) a CDRH3 sequence of SEQ ID NO: 68; and
a light chain variable region comprising:
(d) a CDRL1 sequence of SEQ ID NO: 69; and/or
(e) a CDRL2 sequence of SEQ ID NO: 70; and/or
(f) a CDRL3 sequence of SEQ ID NO: 71.

27. The antibody of claim 26, wherein the CDRH1, CDRH2 and CDRH3 sequences of the second binding unit are present in a human VH framework.

28. The antibody of claim 26 or 27, wherein the CDRL1, CDRL2 and CDRL3 sequences of the second binding unit are present in a human VL framework.

29. The antibody of any one of claims 26-28, wherein the second binding unit comprises:
a heavy chain variable region comprising:
(a) a CDRH1 sequence of SEQ ID NO: 65; and
(b) a CDRH2 sequence of any one of SEQ ID NOs: 66-67; and
(c) a CDRH3 sequence of SEQ ID NO: 68.

30. The antibody of claim 29, wherein the second binding unit comprises:
a heavy chain variable region comprising:
(a) a CDRH1 sequence of SEQ ID NO: 65, a heavy chain CDRH2 sequence of SEQ ID NO: 66, and a heavy chain CDRH3 sequence of SEQ ID NO: 68; or
(b) a CDRH1 sequence of SEQ ID NO: 65, a CDRH2 sequence of SEQ ID NO: 67, and a CDRH3 sequence of SEQ ID NO: 68.

31. The antibody of any one of claims 26-30, wherein the second binding unit comprises:
a light chain variable region comprising: a CDRL1 sequence of SEQ ID NO: 69; a CDRL2 sequence of SEQ ID NO: 70; and a CDRL3 sequence of SEQ ID NO: 71.

32. The antibody of any one of claims 26-31, wherein the second binding unit comprises:
(a) a heavy chain variable region comprising:
a CDRH1 sequence of SEQ ID NO: 65, a CDRH2 sequence of SEQ ID NO: 66, and a CDRH3 sequence of SEQ ID NO: 68; and
a light chain variable region comprising:
a CDRL1 sequence of SEQ ID NO: 69, a CDRL2 sequence of SEQ ID NO: 70, and a CDRL3 sequence of SEQ ID NO: 71; or
(b) a heavy chain variable region comprising:
a CDRH1 sequence of SEQ ID NO: 65, a CDRH2 sequence of SEQ ID NO: 67, and a CDRH3 sequence of SEQ ID NO: 68; and
a light chain variable region comprising:
a CDRL1 sequence of SEQ ID NO: 69, a CDRL2 sequence of SEQ ID NO: 70, and a CDRL3 sequence of SEQ ID NO: 71.

33. The antibody of claim 32, wherein the second binding unit comprises a heavy chain variable region sequence having at least 95% identity to any one of SEQ ID NOs: 72-73.

34. The antibody of claim 32, wherein the second binding unit comprises a heavy chain variable region sequence comprising any one of SEQ ID NOs: 72-73.

35. The antibody of any one of claims 32-34, wherein the second binding unit comprises a light chain variable region sequence having at least 95% identity to any one of SEQ ID NOs: 74-75.

36. The antibody of any one of claims 32-34, wherein the second binding unit comprises a light chain variable region sequence comprising any one of SEQ ID NOs: 74-75.

37. The antibody of any one of claims 32-36, wherein the second binding unit comprises:
(a) a heavy chain variable region sequence of SEQ ID NO: 72 and a light chain variable region sequence of SEQ ID NO: 74; or
(b) a heavy chain variable region sequence of SEQ ID NO: 73 and a light chain variable region sequence of SEQ ID NO: 75.

38. An antibody that binds to CLEC12A and CD38, comprising:
a first light chain subunit comprising SEQ ID NO: 79;
a first heavy chain subunit comprising SEQ ID NO: 76;
a second light chain subunit comprising SEQ ID NO: 79; and
a second heavy chain subunit comprising SEQ ID NO: 78.

39. An antibody that binds to CLEC12A and CD38, comprising:
a first light chain subunit comprising SEQ ID NO: 79;
a first heavy chain subunit comprising SEQ ID NO: 77;
a second light chain subunit comprising SEQ ID NO: 79; and
a second heavy chain subunit comprising SEQ ID NO: 78.

40. An antibody that binds to CLEC12A and CD38, comprising:
a first light chain subunit comprising SEQ ID NO: 83;
a first heavy chain subunit comprising SEQ ID NO: 80;
a second light chain subunit comprising SEQ ID NO: 83; and
a second heavy chain subunit comprising SEQ ID NO: 82.

41. An antibody that binds to CLEC12A and CD38, comprising:
a first light chain subunit comprising SEQ ID NO: 83;
a first heavy chain subunit comprising SEQ ID NO: 81;
a second light chain subunit comprising SEQ ID NO: 83; and
a second heavy chain subunit comprising SEQ ID NO: 82.

42. A pharmaceutical composition comprising the antibody of any one of claims 1-41.

43. A method of treatment, comprising administering to an individual in need an effective dose of the antibody of any one of claims 1-41, or the pharmaceutical composition of claim 42.

44. A method for the treatment of a disorder characterized by expression of CLEC12A, comprising administering to a subject with said disorder the antibody of any one of claims 1-41, or the pharmaceutical composition of claim 42.

45. Use of an antibody of any one of claims 1-41, in the preparation of a medicament for the treatment of a disorder characterized by expression of CLEC12A.

46. An antibody of any one of claims 1-41, for use in the treatment of a disorder characterized by expression of CLEC12A.

47. The method, use, or antibody of any one of claims 44-46, wherein the disorder is a cancer.

48. The method, use, or antibody of claim 47, wherein the cancer is a blood cancer.

49. The method, use, or antibody of claim 48, wherein the blood cancer is acute myeloid leukemia (AML).

50. The method, use, or antibody of claim 49, wherein the AML is refractory AML.

51. A polynucleotide encoding an antibody of any one of claims 1-41.

52. A vector comprising the polynucleotide of claim 51.

53. A cell comprising the vector of claim 52.

54. A method of producing an antibody of any one of claims 1-41, comprising growing a cell according to claim 53 under conditions permissive for expression of the antibody, and isolating the antibody.

55. A kit comprising the antibody of any one of claims 1-41, or the pharmaceutical composition of claim 42, and instructions for use.

56. The kit of claim 55, further comprising an additional therapeutic agent.
